(19) United States
(12) Patent Application Publication
(10) Pub. No.: US 2025/0205351 A1
(43) Pub. Date: Jun. 26, 2025

(54) USE OF AN ANTIBODY-DRUG CONJUGATE IN INHIBITING THE GROWTH OF NON-EGFR-EXPRESSING TUMORS

(71) Applicant: JIANGSU HEALTHY LIFE INNOVATION MEDICAL TECHNOLOGY CO., LTD, Jiangsu (CN)

(72) Inventors: Yuanzhen SUO, Jiangsu (CN); Ying YUE, Jiangsu (CN); Jing YU, Jiangsu (CN); Meixin HAN, Jiangsu (CN); Jingjing FENG, Jiangsu (CN); Yingying WANG, Jiangsu (CN); Jinye YANG, Jiangsu (CN); Yong HUANG, Jiangsu (CN)

(21) Appl. No.: 18/952,146

(22) Filed: Nov. 19, 2024

(30) Foreign Application Priority Data
Dec. 26, 2023 (CN) 202311814141.2
Sep. 25, 2024 (CN) 202411345541.8

(51) Int. Cl. A61K 47/68; A61N 1/36; A61P 35/00

(52) U.S. Cl. CPC A61K 47/68037; A61K 47/68031; A61K 47/6849; A61K 47/6855; A61K 47/6857; A61K 47/6863; A61N 1/36002; A61P 35/00

(57) ABSTRACT

A use of an antibody-drug conjugate in the preparation of medicaments for treating non-EGFR-expressing tumors, wherein the antibody-drug conjugate is administered in combination with an alternating electric fields is disclosed.

### CROSS-REFERENCE TO RELATED PATENT APPLICATIONS

This application claims priority to Chinese application No. 202311814141.2 filed on Dec. 26, 2023 and Chinese application No. 202411345541.8 filed on Sep. 25, 2024, which are herein incorporated by references in their entireties.

### FIELD

The present disclosure relates to the use of an antibody-drug conjugate (ADC) in combination with tumor-treating fields (TTFields) for inhibiting the growth of non-EGFR-expressing tumors.

### BACKGROUND

Antibody-drug conjugates are a rapidly expanding class of cancer therapy, which are formed by linking monoclonal antibodies (mAbs) that target specific antigens with small molecule cytotoxic drugs via linkers. They combine the targeting specificity of the monoclonal antibodies (mAbs) with the cytotoxicity of the potent small molecules. A distinct clinical advantage of ADCs is their ability to deliver toxic payloads directly to a tumor, by passing down stream resistance mechanisms related to intracellular signaling.

Tumor-treating fields (TTFields) are non-invasive treatments that target solid tumors, and are alternating electric fields with low intensity (e.g., 1-3V/cm) and intermediate frequency (e.g., 100-700 kHz). TTFields disrupt cell division through physical interactions that interfere with the assembly of key molecules needed for mitosis. These electric fields are induced non-invasively by arrays of electrodes placed directly onto the patient's scalp.

TTFields are established as an anti-mitotic cancer treatment modality because they interfere with proper microtubule assembly during the metaphase portion of the cell cycle which eventually leads to the destruction of the cells during the telophase and cytokinesis portions of the cell cycle. For cancer treatment, non-invasive devices were developed with capacitively-coupled transducers that are placed directly onto the skin region closest to the tumor. The treatment efficacy increases with increased field strength and the optimal frequency depends on the cancer cell type.

Despite the fact that ADCs have emerged as a type of promising drug for treating both hematological malignancies and solid tumors, with extensive preclinical and clinical research conducted, the duration of objective responses or clinical benefits from ADCs as a monotherapy, like most cytotoxic drugs, remains limited. The combination of ADCs and TTFields in cancer therapy is poised to be a pivotal direction in the treatment of cancer. As revealed in Chinese patent application No. 112543661A, an increased effect of Madparatuzumab in combination with TTFields has demonstrated efficacy in inhibiting the proliferation of glioblastoma.

### SUMMARY

The disclosure provides a use of antibody-drug conjugates in combination with tumor-treating fields for inhibiting the growth of non-EGFR-expressing tumors.

The first aspect of the disclosure provides a method for treating a non-EGFR-expressing tumor, comprising:
applying an alternating electric field to a target area, wherein the target area comprises a non-EGFR-expressing tumor or cancer cell, and
administering an effective amount of an antibody-drug conjugate.

The second aspect of the disclosure provides a method for inhibiting the growth of a non-EGFR-expressing tumor, comprising:
(i) applying an alternating electric field to a target area, wherein the target area comprises a non-EGFR-expressing tumor or cancer cell, and
(ii) administering an effective amount of an antibody-drug conjugate.

The third aspect of the disclosure provides a system for treating a non-EGFR-expressing tumor, comprising:
a patient information processing module and an output module;
the patient information processing module is configured to receive information on patients with the non-EGFR-expressing tumor treated with an antibody-drug conjugate, said information includes at least the medication information of the antibody-drug conjugate administered to said patients with the non-EGFR-expressing tumor; and
the output module is configured to receive information outputted by the patient information processing module and to guide the alternating electric field treatment to said patients with the non-EGFR-expressing tumor.

The fourth aspect of the disclosure provides a method for treating an HER2-expressing tumor, comprising: (i) applying an alternating electric field to a target area, wherein the target area comprises an HER2-expressing tumor or cancer cell, and (ii) administering an effective amount of an antibody-drug conjugate, wherein the antibody-drug conjugate is selected from an antibody-drug conjugate comprising Disitamab Vedotin (such as Aidixi, RC-48), an antibody-drug conjugate comprising Trastuzumab Deruxtecan (such as Enhertu®), an antibody-drug conjugate comprising Trastuzumab Emtansine (such as Kadcyla®), DP-303c, SHR-A1811, SYD985, LCB14-0110, BDC-1001, BB-1701, TAA013, or A166.

The fifth aspect of the disclosure provides a method for treating a TROP-2-expressing tumor, comprising: (i) applying an alternating electric field to a target area, wherein the target area comprises a TROP-2-expressing tumor or cancer cell, and (ii) administering an effective amount of an antibody-drug conjugate, wherein the antibody-drug conjugate is selected from an antibody-drug conjugate comprising Sacituzumab Govitecan (such as Trodelvy®), an antibody-drug conjugate comprising Trastuzumab Deruxtecan (such as Enhertu®), SKB264, an antibody-drug conjugate comprising Detopotamab Deruxtecan, BAT-8008, BL-M02D1, ESG-401, DAC-002, BAT8003, or PF-06664178.

The sixth aspect of the disclosure provides a method for treating an FRα-expressing tumor, comprising: (i) applying an alternating electric field to a target area, wherein the target area comprises an FRα-expressing tumor or cancer cell, and (ii) administering an effective amount of an antibody-drug conjugate, wherein the antibody-drug conjugate is selected from an antibody-drug conjugate comprising Mirvetuximab Soravtansine, STRO-002, PRO-1184, MORAb-202, IMGN-151, or BAT-8006.

The seventh aspect of the disclosure provides a method for treating a TF-expressing tumor, comprising: (i) applying an alternating electric field to a target area, wherein the target area comprises a TF-expressing tumor or cancer cell, and (ii) administering an effective amount of an antibody-drug conjugate, wherein the antibody-drug conjugate is Tivdak®.

The eighth aspect of the disclosure provides a use of an antibody-drug conjugate in the preparation of medicaments for treating a non-EGFR-expressing tumor, wherein the antibody-drug conjugate is administered in combination with an alternating electric field.

The ninth aspect of the disclosure provides a use of an antibody-drug conjugate in the preparation of medicaments for inhibiting the growth of a non-EGFR-expressing tumor, wherein the antibody-drug conjugate is administered in combination with an alternating electric field.

### BRIEF DESCRIPTION OF THE DRAWINGS

FIG. 1A is a three-dimensional assemble view of a TTFields cell experimental device according to an embodiment of the present application.

FIG. 1B is a partial three-dimensional exploded views of the TTFields cell experimental device in FIG. 1A.

FIG. 2 shows the number of cells in the TTFields group at different frequencies in Example 1.

FIG. 3 shows the cytotoxic effect of Enhertu® on NCI-N87 cells in the presence and absence of TTFields.

FIGS. 4A and 4B show the clonogenic effect of Enhertu® on NCI-N87 cells in the presence and absence of TTFields.

FIG. 5 shows the cytotoxic effect of Tivdak® on CaSki cells in the presence and absence of TTFields.

FIGS. 6A and 6B show the clonogenic effect of Tivdak® on CaSki cells in the presence and absence of TTFields.

FIG. 7 shows the cytotoxic effect of Trodelvy® on SK-BR-3 cells in the presence and absence of TTFields.

FIGS. 8A and 8B show the clonogenic effect of Trodelvy® on SK-BR-3 cells in the presence and absence of TTFields.

FIG. 9 shows the cytotoxic effect of Elahere® on SK-OV-3 cells in the presence and absence of TTFields.

FIGS. 10A and 10B show the clonogenic effect of Elahere® on SK-OV-3 cells in the presence and absence of TTFields.

FIG. 11 shows the cytotoxic effect of Enhertu® on SK-BR-3 cells in the presence and absence of TTFields.

FIG. 12 shows the cytotoxic effect of Elahere® on NCI-H1781 cells in the presence and absence of TTFields.

FIGS. 13A and 13B show the clonogenic effect of Elahere® on NCI-H1781 cells in the presence and absence of TTFields.

### DETAILED DESCRIPTION

### Definitions

In order that the disclosure may be more readily understood, certain terms are first defined. In addition, it should be noted that whenever a range of values of a parameter is recited, it is intended that values and ranges intermediate to the recited values are also intended to be part of the present disclosure.

The terms “treat”, “treating” and “treatment” refer to a method of alleviating or abrogating a disease and/or its attendant symptoms.

The term “antibody-drug conjugate” refers to a complex typically composed of an antibody conjugated to a drug molecule via a linker. Such drug molecules can be chemotherapeutic agents, radioactive isotopes, or toxins. The conjugate targets antigens on the surface of specific cells to achieve the therapeutic purposes. As a form of targeted therapy, antibody-drug conjugates are highly selective and specific, capable of treating a variety of diseases including tumors, autoimmune disorders and the like.

The term “antibody” broadly refers to an immunoglobulin (Ig) molecule, generally comprised of four polypeptide chains, two heavy (H) chains, and two light (L) chains. Antibodies comprise complementarity determining regions (CDRs), also known as hypervariable regions, in both the light chain and heavy chain variable domains. The more highly conserved portions of the variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria, while being deemed to be outside a hypervariable region under a different set of criteria. One or more of these positions can also be found in extended hypervariable regions. The variable domains of native heavy and light chains cach comprises four FR regions, largely by adopting a β-sheet configuration, connected by three CDRs, which form loops connecting, and in some cases forming a part of the β-sheet structure. The CDRs in cach chain are held together in close proximity by the FR regions and, with the CDRs from the other chain, contribute to the formation of the antigen binding site of antibodies. See Kabat et al., Sequences of Proteins of Immunological Interest (National Institute of Health, Bethesda, Md. 1987). As used herein, numbering of immunoglobulin amino acid residues is done according to the immunoglobulin amino acid residue numbering system of Kabat et al. unless otherwise indicated.

The term “monoclonal antibody” or “MAb” is not limited to antibodies produced through hybridoma technology. A monoclonal antibody is derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof. In many uses of the present disclosure, including in vivo use of chimeric, primatized, humanized, or human antibodies in humans.

The term “effective amount” or “therapeutically effective amount” refers to the amount of a drug (e.g., an ADC) which is sufficient to reduce or ameliorate the severity and/or duration of a disorder, c.g., cancer, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). The effective amount of an ADC may, for example, inhibit tumor growth (e.g., inhibit an increase in tumor volume), decrease tumor growth (e.g., decrease tumor volume), reduce the number of cancer cells, and/or relieve to some extent one or more of the symptoms associated with the cancer. The effective amount may, for example, improve disease free survival (DFS), improve overall survival (OS), or decrease likelihood of recurrence.

The term “combination” or “combination therapy” refers to the administration of two or more therapies, e.g., ADCs comprising Trastuzumab Deruxtecan and TTFields. The two therapies may be administered concomitantly in which case both therapies are administered together or substantially together, or sequentially in which case one therapy may be administered prior to the other therapy.

The term “non-EGFR-expressing tumors” or “non-EGFR-expressing cancers” refers to tumors or cancers that do not express the epidermal growth factor receptor (EGFR). For example, the “non-EGFR-expressing tumors” or “non-EGFR-expressing cancers” as described in this disclosure refer to breast cancer, gastric cancer, gastroesophageal junction cancer, ovarian epithelial cancer, fallopian tube cancer, primary peritoneal cancer, or cervical cancer, in particular refer to breast cancer, gastric cancer, ovarian cancer, or cervical cancer.

The term “administering” as used herein is meant to refer to the delivery of a substance (e.g., an ADC) to achieve a therapeutic objective. Modes of administration may be parenteral, enteral, and topical. Parenteral administration is usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The term “Tumor Treating Fields” generally refers to the use of alternating electric fields to treat cancer. U.S. Pat. Nos. 6,868,289 and 7,016,725, each of which is incorporated herein by reference in its entirety, disclose methods and apparatuses for treating tumors using alternating electric fields in the range of 1-10 V/cm, at frequencies between 50 kHz and 500 kHz, and that the effectiveness of those fields is increased when more than one field direction is used (e.g., when the field is switched between two or three directions that are oriented about 90° apart from each other).

The first aspect of the disclosure provides a method for treating a non-EGFR-expressing tumor, comprising:
(i) applying an alternating electric field to a target area, wherein the target area comprises a non-EGFR-expressing tumor or cancer cell, and
(ii) administering an effective amount of an antibody-drug conjugate.

The second aspect of the disclosure provides a method for inhibiting the growth of a non-EGFR-expressing tumor, comprising:
(iii) applying an alternating electric field to a target area, wherein the target area comprises a non-EGFR-expressing tumor or cancer cell, and
(iv) administering an effective amount of an antibody-drug conjugate.

The third aspect of the disclosure provides a system for treating a non-EGFR-expressing tumor, comprising:
a patient information processing module and an output module;
the patient information processing module is configured to receive information on patients with the non-EGFR-expressing tumor treated with an antibody-drug conjugate, said information includes at least the medication information of the antibody-drug conjugate administered to said patients with the non-EGFR-expressing tumor; and
the output module is configured to receive information outputted by the patient information processing module and to guide the alternating electric field treatment to said patients with the non-EGFR-expressing tumor.

In some embodiments, the alternating electric field has a frequency of an intermediate frequency range, for example, between 100 kHz and 700 kHz, preferably between 100 kHz and 250 kHz, such as 100 kHz, 150 kHz, 200 kHz, or 250 kHz.

In some embodiments, the alternating electric field has a strength of a low intensity, for example, between 1 V/cm and 3 V/cm, preferably between 1.6 V/cm and 1.8 V/cm, such as 1.6 V/cm, 1.7 V/cm, or 1.8 V/cm.

In some embodiments, the treatment is carried out for more than 48 hours, preferably between 72 and 96 hours, such as 72 hours, 84 hours, or 96 hours.

In some embodiments, the antibody-drug conjugate can be selected from one or more of an antibody-drug conjugate comprising Disitamab Vedotin, an antibody-drug conjugate comprising Trastuzumab Deruxtecan, an antibody-drug conjugate comprising Trastuzumab Emtansine, DP-303c, SHR-A1811, SYD985, LCB14-0110, BDC-1001, BB-1701, TAA013, A166, an antibody-drug conjugate comprising Sacituzumab Govitecan, SKB264, an antibody-drug conjugate comprising Detopotamab Deruxtecan, BAT-8008, BL-M02D1, ESG-401, DAC-002, BAT8003, PF-06664178, an antibody-drug conjugate comprising Mirvetuximab Soravtansine, STRO-002, PRO-1184, MORAb-202, IMGN-151, BAT-8006, an antibody-drug conjugate comprising Tisotumab, an antibody-drug conjugate comprising Gemtuzumab, an antibody-drug conjugate comprising Enfortumab Vedotin-ejfv, an antibody-drug conjugate comprising Brentuximab Vedotin, an antibody-drug conjugate comprising Loncastuximab Tesirine, an antibody-drug conjugate comprising Tisotumab Vedotin, an antibody-drug conjugate Inotuzumab Ozogamicin, an antibody-drug conjugate comprising Belantamab Mafodotin, and an antibody-drug conjugate comprising Cetuximab.

In some embodiments, the antibody-drug conjugate can be selected from Enhertu®, Tivdak®, Trodelvy® or Elahere®.

In some embodiments, the non-EGFR-expressing tumor can be selected from one or more of HER2-expressing tumors, TROP-2-expressing tumors, TF-expressing tumors, Claudin 18.2-expressing tumors, TOP-1-expressing tumors, FRα-expressing tumors, CD33-expressing tumors, CD22-expressing tumors, CEACAM-5-expressing tumors, Mesothelin-expressing tumors, NECTIN4-expressing tumors, LIV1 (SLC39A6/ZIP6)-expressing tumors, ROR1-expressing tumors, GUC2C (GC-C)-expressing tumors, BCMA-expressing tumors, c-Met-expressing tumors, LRRC15-expressing tumors, LY75 (CD205)-expressing tumors, ENPP3-expressing tumors, TIM1-expressing tumors, PTK7-expressing tumors, CD142-expressing tumors, Ax1-expressing tumors, CD276-expressing tumors, CA125 (MUC16)-expressing tumors, CD70-expressing tumors, CD19-expressing tumors, CD30-expressing tumors, CD37-expressing tumors, CD46-expressing tumors, CD48-expressing tumors, CD74-expressing tumors, CD79b-expressing tumors, DLL3-expressing tumors, EpCAM-expressing tumors, ErbB3-expressing tumors, FLT3-expressing tumors, HGF/R-expressing tumors, IGF-IR-expressing tumors, CD25-expressing tumors, CD123-expressing tumors, CD56-expressing tumors, PSMA/FOLH1-expressing tumors, ROR2-expressing tumors, SLCIA5-expressing tumors, CD138-expressing tumors, TNFa-expressing tumors, TPBG-expressing tumors, TRAIL R2 (TNFRSF10B)-expressing tumors, and CD71-expressing tumors.

In some embodiments, the non-EGFR-expressing tumor can be selected from breast cancer, gastric cancer, gastroesophageal junction cancer, HER2-mutated metastatic or unresectable non-small cell lung cancer, ovarian epithelial cancer, fallopian tube cancer, primary peritoneal cancer, or cervical cancer, in particular breast cancer, gastric cancer, HER2-mutated metastatic or unresectable non-small cell lung cancer, ovarian cancer, or cervical cancer.

In some embodiments, the medication information includes, but is not limited to, the type of antibody-drug conjugate, the method of administration, the start and end times of administration, and one or more side effects after the administration.

The fourth aspect of the disclosure provides a method for treating an HER2-expressing tumor, comprising: (i) applying an alternating electric field to a target area, wherein the target area comprises an HER2-expressing tumor or cancer cell, and (ii) administering an effective amount of an antibody-drug conjugate, wherein the antibody-drug conjugate is selected from an antibody-drug conjugate comprising Disitamab Vedotin (such as Aidixi, RC-48), an antibody-drug conjugate comprising Trastuzumab Deruxtecan (such as Enhertu®), an antibody-drug conjugate comprising Trastuzumab Emtansine (such as Kadcyla®), DP-303c, SHR-A1811, SYD985, LCB14-0110, BDC-1001, BB-1701, TAA013, or A166.

In some embodiments, the HER2-expressing tumor is breast cancer, gastric cancer, gastroesophageal junction cancer, HER2-mutated metastatic or unresectable non-small cell lung cancer, in particular gastric cancer.

In some embodiments, the antibody-drug conjugate is Enhertu®.

The fifth aspect of the disclosure provides a method for treating a TROP-2-expressing tumor, comprising: (i) applying an alternating electric field to a target area, wherein the target area comprises a TROP-2-expressing tumor or cancer cell, and (ii) administering an effective amount of an antibody-drug conjugate, wherein the antibody-drug conjugate is selected from an antibody-drug conjugate comprising Sacituzumab Govitecan (such as Trodelvy®), an antibody-drug conjugate comprising Trastuzumab Deruxtecan (such as Enhertu®), SKB264, an antibody-drug conjugate comprising Detopotamab Deruxtecan, BAT-8008, BL-M02D1, ESG-401, DAC-002, BAT8003, or PF-06664178.

In some embodiments, the TROP-2-expressing tumor is breast cancer, in particular triple-negative breast cancer.

In some embodiments, the antibody-drug conjugate is Trodelvy® or Enhertu®.

The sixth aspect of the disclosure provides a method for treating an FRα-expressing tumor, comprising: (i) applying an alternating electric field to a target area, wherein the target area comprises an FRα-expressing tumor or cancer cell, and (ii) administering an effective amount of an antibody-drug conjugate, wherein the antibody-drug conjugate is selected from an antibody-drug conjugate comprising Mirvetuximab Soravtansine, STRO-002, PRO-1184, MORAb-202, IMGN-151, or BAT-8006.

In some embodiments, the FRα-expressing tumor is ovarian epithelial cancer, fallopian tube cancer, or primary peritoneal cancer, in particular ovarian cancer.

In some embodiments, the antibody-drug conjugate is Elahere®.

The seventh aspect of the disclosure provides a method for treating a TF-expressing tumor, comprising: (i) applying an alternating electric field to a target area, wherein the target area comprises a TF-expressing tumor or cancer cell, and (ii) administering an effective amount of an antibody-drug conjugate, wherein the antibody-drug conjugate is Tivdak®.

In some embodiments, the TF-expressing tumor is cervical cancer.

In some embodiments, the alternating electric field has a frequency of an intermediate frequency range, for example, between 100 kHz and 700 kHz, preferably between 100 kHz and 250 kHz.

In some embodiments, the alternating electric field has a strength of a low intensity, for example, between 1 V/cm and 3 V/cm, preferably between 1.6 V/cm and 1.8 V/cm.

The eighth aspect of the disclosure provides a use of an antibody-drug conjugate in the preparation of medicaments for treating a non-EGFR-expressing tumor, wherein the antibody-drug conjugate is administered in combination with an alternating electric field.

The ninth aspect of the disclosure provides a use of an antibody-drug conjugate in the preparation of medicaments for inhibiting the growth of a non-EGFR-expressing tumor, wherein the antibody-drug conjugate is administered in combination with an alternating electric field.

In some aspects, the antibody-drug conjugate for use in treating an HER2-expressing tumor can be administered in combination with an alternating electric field. The antibody-drug conjugate for use in treating an HER2-expressing tumor includes, but is not limited to, an antibody-drug conjugate comprising Disitamab Vedotin (such as Aidixi, RC-48), an antibody-drug conjugate comprising Trastuzumab Deruxtecan (such as Enhertu®), an antibody-drug conjugate comprising Trastuzumab Emtansine (such as Kadcyla®), DP-303c, SHR-A1811, SYD985, LCB14-0110, BDC-1001, BB-1701, TAA013, or A166.

In some aspects, the antibody-drug conjugate for use in treating a TF-expressing tumor can be administered in combination with an alternating electric field. The antibody-drug conjugate for use in treating a TF-expressing tumor includes, but is not limited to, an antibody-drug conjugate comprising Tisotumab (such as tisotumab vedotin-tftv, Tivdak®).

In some aspects, the antibody-drug conjugate for use in treating a TROP-2-expressing tumor can be administered in combination with an alternating electric field. The antibody-drug conjugate for use in treating a TROP-2-expressing tumor includes, but is not limited to, an antibody-drug conjugate comprising Sacituzumab Govitecan (such as Trodelvy®), an antibody-drug conjugate comprising Trastuzumab Deruxtecan (such as Enhertu®), SKB264, an antibody-drug conjugate comprising Detopotamab Deruxtecan, BAT-8008, BL-M02D1, ESG-401, DAC-002, BAT8003, or PF-06664178.

In some aspects, the antibody-drug conjugate for use in treating a Claudin 18.2-expressing tumor can be administered in combination with an alternating electric field. The antibody-drug conjugate for use in treating a Claudin 18.2-expressing tumor includes, but is not limited to, LM-302, RC118, SYSA-1801, JS-107, CMG901 or IBI-343.

In some aspects, the antibody-drug conjugate for use in treating a TOP-1-expressing tumor can be administered in combination with an alternating electric field. The antibody-drug conjugate for use in treating a TOP-1-expressing tumor includes, but is not limited to, an antibody-drug conjugate comprising Sacituzumab Govitecan (such as Trodelvy®).

In some aspects, the antibody-drug conjugate for use in treating an FRα-expressing tumor can be administered in combination with an alternating electric field. The antibody-drug conjugate for use in treating an FRα-expressing tumor includes, but is not limited to, an antibody-drug conjugate comprising Mirvetuximab Soravtansine, STRO-002, PRO-1184, MORAb-202, IMGN-151, or BAT-8006.

In some aspects, the antibody-drug conjugate for use in treating a CD33-expressing tumor can be administered in combination with an alternating electric field. The antibody-drug conjugate for use in treating a CD33-expressing tumor includes, but is not limited to, an antibody-drug conjugate comprising Gemtuzumab Ozogamicin, 225Ac-lintuzumab or DXC-007.

In some aspects, the antibody-drug conjugate for use in treating a CD22-expressing tumor can be administered in combination with an alternating electric field. The antibody-drug conjugate for use in treating a CD22-expressing tumor includes, but is not limited to, an antibody-drug conjugate comprising Inotuzumab Ozogamicin or TAC-001.

In some aspects, the antibody-drug conjugate for use in treating a CEACAM-5-expressing tumor can be administered in combination with an alternating electric field. The antibody-drug conjugate for use in treating a CEACAM-5-expressing tumor includes, but is not limited to, an antibody-drug conjugate comprising Tusamitamab Ravtansine, M-9140 or an antibody-drug conjugate comprising Labetuzumab Govitecan.

In some aspects, the antibody-drug conjugate for use in treating a Mesothelin-expressing tumor can be administered in combination with an alternating electric field. The antibody-drug conjugate for use in treating a Mesothelin-expressing tumor includes, but is not limited to, RC88 or an antibody-drug conjugate comprising Anctumab Ravtansine.

In some aspects, the antibody-drug conjugate for use in treating a NECTIN4-expressing tumor can be administered in combination with an alternating electric field. The antibody-drug conjugate for use in treating a NECTIN4-expressing tumor includes, but is not limited to, an antibody-drug conjugate comprising Enfortumab Vedotin-cjfv, CRB-701, 9MW-2821 or SBT-6290.

In some aspects, the antibody-drug conjugate for use in treating a LIV1 (SLC39A6/ZIP6)-expressing tumor can be administered in combination with an alternating electric field. The antibody-drug conjugate for use in treating a LIV1 (SLC39A6/ZIP6)-expressing tumor includes, but is not limited to, an antibody-drug conjugate comprising Ladiratuzumab Vedotin.

In some aspects, the antibody-drug conjugate for use in treating a ROR1-expressing tumor can be administered in combination with an alternating electric field. The antibody-drug conjugate for use in treating a ROR1-expressing tumor includes, but is not limited to, an antibody-drug conjugate comprising Zilovertamab Vedotin, LCB-71 or NBE-002.

In some aspects, the antibody-drug conjugate for use in treating a GUC2C (GC-C)-expressing tumor can be administered in combination with an alternating electric field. The antibody-drug conjugate for use in treating a GUC2C (GC-C)-expressing tumor includes, but is not limited to, TAK-164.

In some aspects, the antibody-drug conjugate for use in treating a BCMA-expressing tumor can be administered in combination with an alternating electric field. The antibody-drug conjugate for use in treating a BCMA-expressing tumor includes, but is not limited to, an antibody-drug conjugate comprising Belantamab Mafodotin.

In some aspects, the antibody-drug conjugate for use in treating a c-Met-expressing tumor can be administered in combination with an alternating electric field. The antibody-drug conjugate for use in treating a c-Met-expressing tumor includes, but is not limited to, RC108 or an antibody-drug conjugate comprising Telisotuzumab Vedotin.

In some aspects, the antibody-drug conjugate for use in treating a LY75 (CD205)-expressing tumor can be administered in combination with an alternating electric field. The antibody-drug conjugate for use in treating a LY75 (CD205)-expressing tumor includes, but is not limited to, MEN-1309.

In some aspects, the antibody-drug conjugate for use in treating an ENPP3-expressing tumor can be administered in combination with an alternating electric field. The antibody-drug conjugate for use in treating an ENPP3-expressing tumor includes, but is not limited to, AGS-16C3F.

In some aspects, the antibody-drug conjugate for use in treating a PTK7-expressing tumor can be administered in combination with an alternating electric field. The antibody-drug conjugate for use in treating a PTK7-expressing tumor includes, but is not limited to, an antibody-drug conjugate comprising Cofetuzumab Pelidotin.

In some aspects, the antibody-drug conjugate for use in treating a CD142-expressing tumor can be administered in combination with an alternating electric field. The antibody-drug conjugate for use in treating a CD142-expressing tumor includes, but is not limited to, an antibody-drug conjugate comprising Tisotumab Vedotin or MRG-004A.

In some aspects, the antibody-drug conjugate for use in treating an Ax1-expressing tumor can be administered in combination with an alternating electric field. The antibody-drug conjugate for use in treating an Ax1-expressing tumor includes, but is not limited to, BA-3011 or an antibody-drug conjugate comprising Mipasetamab Uzoptirine.

In some aspects, the antibody-drug conjugate for use in treating a CD276-expressing tumor can be administered in combination with an alternating electric field. The antibody-drug conjugate for use in treating a CD276-expressing tumor includes, but is not limited to, 7MW3711, HS-20093, DS-7300, MGC-018 or BAT-8009.

In some aspects, the antibody-drug conjugate for use in treating a CA125 (MUC16)-expressing tumor can be administered in combination with an alternating electric field. The antibody-drug conjugate for use in treating a CA125 (MUC16)-expressing tumor includes, but is not limited to, RG-7882 or an antibody-drug conjugate comprising Sofituzumab Vedotin.

In some aspects, the antibody-drug conjugate for use in treating a CD70-expressing tumor can be administered in combination with an alternating electric field. The antibody-drug conjugate for use in treating a CD70-expressing tumor includes, but is not limited to, PRO-1160, ARX-305, AMG-172, SGN-CD70A and an antibody-drug conjugate comprising Vorsetuzumab Mafodotin, MDX-1203 or MDX-1411.

In some aspects, the antibody-drug conjugate for use in treating a CD19-expressing tumor can be administered in combination with an alternating electric field. The antibody-drug conjugate for use in treating a CD19-expressing tumor includes, but is not limited to, Lonca.

In some aspects, the antibody-drug conjugate for use in treating a CD30-expressing tumor can be administered in combination with an alternating electric field. The antibody-drug conjugate for use in treating a CD30-expressing tumor includes, but is not limited to, an antibody-drug conjugate comprising Brentuximab Vedotin, or F0002-ADC.

In some aspects, the antibody-drug conjugate for use in treating a CD37-expressing tumor can be administered in combination with an alternating electric field. The antibody-drug conjugate for use in treating a CD37-expressing tumor includes, but is not limited to, an antibody-drug conjugate comprising Lutetium (177Lu) Lilotomab Satetraxetan, AGS-67E or DEBIO-1562.

In some aspects, the antibody-drug conjugate for use in treating a CD46-expressing tumor can be administered in combination with an alternating electric field. The antibody-drug conjugate for use in treating a CD46-expressing tumor includes, but is not limited to, FOR46.

In some aspects, the antibody-drug conjugate for use in treating a CD48-expressing tumor can be administered in combination with an alternating electric field. The antibody-drug conjugate for use in treating a CD48-expressing tumor includes, but is not limited to, SGN-CD48A.

In some aspects, the antibody-drug conjugate for use in treating a CD74-expressing tumor can be administered in combination with an alternating electric field. The antibody-drug conjugate for use in treating a CD74-expressing tumor includes, but is not limited to, STRO-001.

In some aspects, the antibody-drug conjugate for use in treating a CD79b-expressing tumor can be administered in combination with an alternating electric field. The antibody-drug conjugate for use in treating a CD79b-expressing tumor includes, but is not limited to, an antibody-drug conjugate comprising Polatuzumab Vedotin, NBT-508 or DCDS-0780A.

In some aspects, the antibody-drug conjugate for use in treating a DLL3-expressing tumor can be administered in combination with an alternating electric field. The antibody-drug conjugate for use in treating a DLL3-expressing tumor includes, but is not limited to, an antibody-drug conjugate comprising Rovalpituzumab Tesirine.

In some aspects, the antibody-drug conjugate for use in treating an ErbB3-expressing tumor can be administered in combination with an alternating electric field. The antibody-drug conjugate for use in treating an ErbB3-expressing tumor includes, but is not limited to, BL-B01D1 or an antibody-drug conjugate comprising Patritumab Deruxtecan.

In some aspects, the antibody-drug conjugate for use in treating an IGF-IR-expressing tumor can be administered in combination with an alternating electric field. The antibody-drug conjugate for use in treating an IGF-IR-expressing tumor includes, but is not limited to, W-0101.

In some aspects, the antibody-drug conjugate for use in treating a CD25-expressing tumor can be administered in combination with an alternating electric field. The antibody-drug conjugate for use in treating a CD25-expressing tumor includes, but is not limited to, an antibody-drug conjugate comprising Camidanlumab Tesirine or LMB-2.

In some aspects, the antibody-drug conjugate for use in treating a CD123-expressing tumor can be administered in combination with an alternating electric field. The antibody-drug conjugate for use in treating a CD123-expressing tumor includes, but is not limited to, IMGN-632 or SGN-CD123A.

In some aspects, the antibody-drug conjugate for use in treating a CD56-expressing tumor can be administered in combination with an alternating electric field. The antibody-drug conjugate for use in treating a CD56-expressing tumor includes, but is not limited to, an antibody-drug conjugate comprising Lorvotuzumab Mertansine.

In some aspects, the antibody-drug conjugate for use in treating a ROR2-expressing tumor can be administered in combination with an alternating electric field. The antibody-drug conjugate for use in treating a ROR2-expressing tumor includes, but is not limited to, an antibody-drug conjugate comprising Ozuriftamab.

In some aspects, the antibody-drug conjugate for use in treating a CD138-expressing tumor can be administered in combination with an alternating electric field. The antibody-drug conjugate for use in treating a CD138-expressing tumor includes, but is not limited to, an antibody-drug conjugate comprising Indatuximab Ravtansine.

In some aspects, the antibody-drug conjugate for use in treating a TPBG-expressing tumor can be administered in combination with an alternating electric field. The antibody-drug conjugate for use in treating a TPBG-expressing tumor includes, but is not limited to, ASN-004, SYD-1875, an antibody-drug conjugate comprising Naptumomab Estafenatox or PF-06263507.

In some aspects, the antibody-drug conjugate for use in treating a CD71-expressing tumor can be administered in combination with an alternating electric field. The antibody-drug conjugate for use in treating a CD71-expressing tumor includes, but is not limited to, an antibody-drug conjugate comprising CX-2029.

In some embodiments, the antibody-drug conjugate comprising Trastuzumab Deruxtecan is administered in combination with TTFields having a frequency between 100 kHz and 250 kHz and a field strength between 1.6V/cm and 1.8V/cm to HER2-positive gastric cancer cell line NCI-N87 (the type of tumor cells is consistent with the approved indication of the antibody-drug conjugate) in the present application to test the efficacy of the antibody-drug conjugate in combination with TTFields through cytotoxic effect and clonogenic effect experiments.

In some embodiments, the present application also provides a medication regimen for inhibiting gastric cancer by combining an antibody-drug conjugate comprising Trastuzumab Deruxtecan with TTFields, comprising: applying an alternating electric field with a certain frequency and field strength to the gastric target area of the subject for a period of time; and administering the recommended dose of an antibody-drug conjugate comprising Trastuzumab Deruxtecan to the gastric target area of the subject in need. Specifically, the alternating electric field can be applied before, after, or simultaneously with the administration of the antibody-drug conjugate comprising Trastuzumab Deruxtecan. Alternatively, the antibody-drug conjugate comprising Trastuzumab Deruxtecan can be administered before, after, or simultaneously with the application of the alternating electric field. Applying the alternating electric field before administering the antibody-drug conjugate comprising Trastuzumab Deruxtecan can include applying the alternating electric field several seconds, minutes, hours, or days before administering the antibody-drug conjugate comprising Trastuzumab Deruxtecan. Applying the alternating electric field after administering the antibody-drug conjugate comprising Trastuzumab Deruxtecan can include applying the alternating electric field several seconds, minutes, hours, or days after administering the antibody-drug conjugate comprising Trastuzumab Deruxtecan. Applying the alternating electric field simultaneously with administering the antibody-drug conjugate comprising Trastuzumab Deruxtecan can include applying the alternating electric field several seconds or minutes before or after administering the antibody-drug conjugate comprising Trastuzumab Deruxtecan. Simultaneously applying the alternating electric field and administering the antibody-drug conjugate comprising Trastuzumab Deruxtecan can include applying the alternating electric field while administering the antibody-drug conjugate comprising Trastuzumab Deruxtecan. Preferably, the frequency of the alternating electric field is 150 kHz, and the strength is 1.6V/cm; the recommended dose of the antibody-drug conjugate comprising Trastuzumab Deruxtecan is 6.4 mg/kg for intravenous drip once every 3 weeks (21-day course) until disease progresses or unacceptable toxicity occurs.

In some embodiments, the antibody-drug conjugate comprising Trastuzumab Deruxtecan is administered in combination with TTFields having a frequency between 100 kHz and 250 kHz and a field strength between 1.6 V/cm and 1.8 V/cm to Trop-2-positive breast cancer cells SK-BR-3 (the type of tumor cells is consistent with the approved indication of the antibody-drug conjugate) in the present application to test the efficacy of the antibody-drug conjugate in combination with TTFields through cytotoxic effect and clonogenic effect experiments.

In some embodiments, the present application also provides a medication regimen for inhibiting breast cancer by combining an antibody-drug conjugate comprising Trastuzumab Deruxtecan with TTFields, comprising: applying an alternating electric field with a certain frequency and field strength to the breast target area of the subject for a period of time; and administering the recommended dose of an antibody-drug conjugate comprising Trastuzumab Deruxtecan to the breast target area of the subject in need. Specifically, the alternating electric field can be applied before, after, or simultaneously with the administration of the antibody-drug conjugate comprising Trastuzumab Deruxtecan. Alternatively, the antibody-drug conjugate comprising Trastuzumab Deruxtecan can be administered before, after, or simultaneously with the application of the alternating electric field. Applying the alternating electric field before administering the antibody-drug conjugate comprising Trastuzumab Deruxtecan can include applying the alternating electric field several seconds, minutes, hours, or days before administering the antibody-drug conjugate comprising Trastuzumab Deruxtecan. Applying the alternating electric field after administering the antibody-drug conjugate comprising Trastuzumab Deruxtecan can include applying the alternating electric field several seconds, minutes, hours, or days after administering the antibody-drug conjugate comprising Trastuzumab Deruxtecan. Applying the alternating electric field simultaneously with administering the antibody-drug conjugate comprising Trastuzumab Deruxtecan can include applying the alternating electric field several seconds or minutes before or after administering the antibody-drug conjugate comprising Trastuzumab Deruxtecan. Simultaneously applying the alternating electric field and administering the antibody-drug conjugate comprising Trastuzumab Deruxtecan can include applying the alternating electric field while administering the antibody-drug conjugate comprising Trastuzumab Deruxtecan. Preferably, the frequency of the alternating electric field is 150 kHz, and the strength is 1.7V/cm; the recommended dose of the antibody-drug conjugate comprising Trastuzumab Deruxtecan is 5.4 mg/kg for intravenous drip once every 3 weeks (21-day course) until disease progresses or unacceptable toxicity occurs.

In some embodiments, the antibody-drug conjugate comprising Trastuzumab Deruxtecan is administered in combination with TTFields having a frequency between 100 kHz and 250 kHz and a field strength between 1.6 V/cm and 1.8 V/cm to HER2-mutated non-small cell lung cancer cells NCI-H1781 (the type of tumor cells is consistent with the approved indication of the antibody-drug conjugate) in the present application to test the efficacy of the antibody-drug conjugate in combination with TTFields through cytotoxic effect and clonogenic effect experiments.

In some embodiments, the present application also provides a medication regimen for inhibiting HER2-mutated metastatic or unresectable non-small cell lung cancer by combining an antibody-drug conjugate comprising Trastuzumab Deruxtecan with TTFields, comprising: applying an alternating electric field with a certain frequency and field strength to the lung target area of the subject for a period of time; and administering the recommended dose of an antibody-drug conjugate comprising Trastuzumab Deruxtecan to the lung target area of the subject in need. Specifically, the alternating electric field can be applied before, after, or simultaneously with the administration of the antibody-drug conjugate comprising Trastuzumab Deruxtecan. Alternatively, the antibody-drug conjugate comprising Trastuzumab Deruxtecan can be administered before, after, or simultaneously with the application of the alternating electric field. Applying the alternating electric field before administering the antibody-drug conjugate comprising Trastuzumab Deruxtecan can include applying the alternating electric field several seconds, minutes, hours, or days before administering the antibody-drug conjugate comprising Trastuzumab Deruxtecan. Applying the alternating electric field after administering the antibody-drug conjugate comprising Trastuzumab Deruxtecan can include applying the alternating electric field several seconds, minutes, hours, or days after administering the antibody-drug conjugate comprising Trastuzumab Deruxtecan. Applying the alternating electric field simultaneously with administering the antibody-drug conjugate comprising Trastuzumab Deruxtecan can include applying the alternating electric field several seconds or minutes before or after administering the antibody-drug conjugate comprising Trastuzumab Deruxtecan. Simultaneously applying the alternating electric field and administering the antibody-drug conjugate comprising Trastuzumab Deruxtecan can include applying the alternating electric field while administering the antibody-drug conjugate comprising Trastuzumab Deruxtecan. Preferably, the frequency of the alternating electric field is 150 kHz, and the strength is 1.8 V/cm; the recommended dose of the antibody-drug conjugate comprising Trastuzumab Deruxtecan is 5.4 mg/kg for intravenous drip once every 3 weeks (21-day course) until disease progresses or unacceptable toxicity occurs.

In some embodiments, the antibody-drug conjugate comprising Trastuzumab Deruxtecan can be Enhertu®.

Enhertu® (Trastuzumab Deruxtecan (T-DXd); DS-8201) is an antibody-drug conjugate developed jointly by AstraZeneca and Daiichi Sankyo. Enhertu is an antibody-drug conjugate that links a humanized monoclonal antibody targeting HER2, trastuzumab, with a new type of small molecule toxic drug, a derivative of topoisomerase 1 inhibitor exatecan (a derivative of DX-8951, DXd), via a 4-peptide linker. After binding to HER2 on tumor cells, T-DXd undergoes internalization and intracellular linker cleavage by lysosomal enzymes. Upon release, the membrane-permeable DXd enters the cell nucleus, causing DNA damage and cell death. Enhertu® has been approved globally for several cancer indications, including HER2-positive breast cancer, gastric cancer, and gastroesophageal junction cancer.

In some embodiments, the antibody-drug conjugate comprising Tisotumab is administered in combination with TTFields having a frequency between 100 kHz and 250 kHz and a field strength between 1.6 V/cm and 1.8 V/cm to high TF-expressing cervical cancer cell line CaSki (the type of tumor cells is consistent with the approved indication of the antibody-drug conjugate) in the present application to test the efficacy of the antibody-drug conjugate in combination with TTFields through cytotoxic effect and clonogenic effect experiments.

In some embodiments, the present application also provides a medication regimen for inhibiting cervical cancer by combining an antibody-drug conjugate comprising Tisotumab with TTFields, comprising: applying an alternating electric field with a certain frequency and field strength to the cervical target area of the subject for a period of time; and administering the recommended dose of an antibody-drug conjugate comprising Tisotumab to the cervical target area of the subject in need. Specifically, the alternating electric field can be applied before, after, or simultaneously with the administration of the antibody-drug conjugate comprising Tisotumab. Alternatively, the antibody-drug conjugate comprising Tisotumab can be administered before, after, or simultaneously with the application of the alternating electric field. Applying the alternating electric field before administering the antibody-drug conjugate comprising Tisotumab can include applying the alternating electric field several seconds, minutes, hours, or days before administering the antibody-drug conjugate comprising Tisotumab. Applying the alternating electric field after administering the antibody-drug conjugate comprising Tisotumab can include applying the alternating electric field several seconds, minutes, hours, or days after administering the antibody-drug conjugate comprising Tisotumab. Applying the alternating electric field simultaneously with administering the antibody-drug conjugate comprising Tisotumab can include applying the alternating electric field several seconds or minutes before or after administering the antibody-drug conjugate comprising Tisotumab. Simultaneously applying the alternating electric field and administering the antibody-drug conjugate comprising Tisotumab can include applying the alternating electric field while administering the antibody-drug conjugate comprising Tisotumab. Preferably, the frequency of the alternating electric field is 200 kHz, and the strength is 1.7 V/cm; the recommended dose of the antibody-drug conjugate comprising Tisotumab is 2 mg/kg (for patients with a body weight ≥ 100 kg, the maximum dose is 200 mg) for intravenous infusion 30 minutes every 3 weeks until disease progresses or unacceptable toxicity occurs.

In some embodiments, the antibody-drug conjugate comprising Tisotumab can be Tivdak®.

Tivdak® (tisotumab vedotin-tftv; TF-011-MMAE) is an antibody-drug conjugate jointly developed by Seagen and Genmab, linking the small molecule toxic drug monomethyl auristatin E to an anti-TF monoclonal antibody TF-011 via a protease-cleavable linker. The anti-cancer activity of Tivdak® is due to the binding of the ADC to TF-expressing cancer cells, followed by internalization of the ADC-TF complex and release of MMAE through proteolytic cleavage. MMAE disrupts the microtubule network in actively dividing cells, leading to cell cycle arrest and apoptotic cell death. Tivdak® also mediates antibody-dependent cellular phagocytosis and antibody-dependent cellular cytotoxicity in vitro. On Sep. 20, 2021, Tivdak® was granted an accelerated approval by the U.S. FDA for the treatment of adult patients with recurrent or metastatic cervical cancer whose disease progresses during or after chemotherapy.

In some embodiments, the antibody-drug conjugate comprising Sacituzumab Govitecan is administered in combination with TTFields having a frequency between 100 kHz and 250 kHz and a field strength between 1.6 V/cm and 1.8 V/cm to Trop-2-positive breast cancer cells SK-BR-3 (the type of tumor cells is consistent with the approved indication of the antibody-drug conjugate) in the present application to test the efficacy of the antibody-drug conjugate in combination with TTFields through cytotoxic effect and clonogenic effect experiments.

In some embodiments, the present application also provides a medication regimen for inhibiting breast cancer by combining an antibody-drug conjugate comprising Sacituzumab Govitecan with TTFields, comprising: applying an alternating electric field with a certain frequency and field strength to the breast target area of the subject for a period of time; and administering the recommended dose of an antibody-drug conjugate comprising Sacituzumab Govitecan to the breast target area of the subject in need. Specifically, the alternating electric field can be applied before, after, or simultaneously with the administration of the antibody-drug conjugate comprising Sacituzumab Govitecan. Alternatively, the antibody-drug conjugate comprising Sacituzumab Govitecan can be administered before, after, or simultaneously with the application of the alternating electric field. Applying the alternating electric field before administering the antibody-drug conjugate comprising Sacituzumab Govitecan can include applying the alternating electric field several seconds, minutes, hours, or days before administering the antibody-drug conjugate comprising Sacituzumab Govitecan. Applying the alternating electric field after administering the antibody-drug conjugate comprising Sacituzumab Govitecan can include applying the alternating electric field several seconds, minutes, hours, or days after administering the antibody-drug conjugate comprising Sacituzumab Govitecan. Applying the alternating electric field simultaneously with administering the antibody-drug conjugate comprising Sacituzumab Govitecan can include applying the alternating electric field several seconds or minutes before or after administering the antibody-drug conjugate comprising Sacituzumab Govitecan. Simultaneously applying the alternating electric field and administering the antibody-drug conjugate comprising Sacituzumab Govitecan can include applying the alternating electric field while administering the antibody-drug conjugate comprising Sacituzumab Govitecan. Preferably, the frequency of the alternating electric field is 150 kHz, and the strength is 1.6 V/cm; the recommended dose of the antibody-drug conjugate comprising Sacituzumab Govitecan is 10 mg/kg for intravenous infusion only, once on Day 1 and Day 8 of a continuous 21-day treatment course, until disease progresses or unacceptable toxicity occurs.

In some embodiments, the antibody-drug conjugate comprising Sacituzumab Govitecan can be Trodelvy®.

Trodelvy® (Sacituzumab Govitecan; IMMU-132) is an antibody-drug conjugate developed by Immunomedics. Trodelvy is formed by linking a Trop-2 monoclonal antibody sacituzumab with a small molecule toxic drug SN-38 via a linker CL2A. Trop-2 is generally highly expressed in triple-negative breast cancer, promoting the growth of cancer cells. The small molecule toxic drug SN-38 is a topoisomerase I inhibitor that is connected to the antibody via the hydrolyzable linker CL2A. Once Sacituzumab Govitecan binds to the Trop-2 receptor and enters the cell, the small molecule toxic drug SN-38 is released through the hydrolysis of the linker and inhibits DNA topoisomerase I, causing frequent single-strand DNA breaks, and ultimately leading to cell death. On Apr. 22, 2020, Trodelvy® was approved in the U.S. for the treatment of adult patients with metastatic triple-negative breast cancer who have received at least two prior therapies. Trodelvy® is the first globally approved antibody-drug conjugate targeting TROP-2.

In some embodiments, the antibody-drug conjugate comprising Mirvetuximab Soravtansine is administered in combination with TTFields having a frequency between 100 kHz and 250 kHz and a field strength between 1.6 V/cm and 1.8 V/cm to high FRα-expressing ovarian cancer cell line SK-OV-3 (the type of tumor cells is consistent with the approved indication of the antibody-drug conjugate) in the present application to test the efficacy of the antibody-drug conjugate in combination with TTFields through cytotoxic effect and clonogenic effect experiments.

In some embodiments, the present application also provides a medication regimen for inhibiting ovarian cancer by combining an antibody-drug conjugate comprising Mirvetuximab Soravtansine with TTFields, comprising: applying an alternating electric field with a certain frequency and field strength to the ovarian target area of the subject for a period of time; and administering the recommended dose of an antibody-drug conjugate comprising Mirvetuximab Soravtansine to the ovarian target area of the subject in need. Specifically, the alternating electric field can be applied before, after, or simultaneously with the administration of the antibody-drug conjugate comprising Mirvetuximab Soravtansine. Alternatively, the antibody-drug conjugate comprising Mirvetuximab Soravtansine can be administered before, after, or simultaneously with the application of the alternating electric field. Applying the alternating electric field before administering the antibody-drug conjugate comprising Mirvetuximab Soravtansine can include applying the alternating electric field several seconds, minutes, hours, or days before administering the antibody-drug conjugate comprising Mirvetuximab Soravtansine. Applying the alternating electric field after administering the antibody-drug conjugate comprising Mirvetuximab Soravtansine can include applying the alternating electric field several seconds, minutes, hours, or days after administering the antibody-drug conjugate comprising Mirvetuximab Soravtansine. Applying the alternating electric field simultaneously with administering the antibody-drug conjugate comprising Mirvetuximab Soravtansine can include applying the alternating electric field several seconds or minutes before or after administering the antibody-drug conjugate comprising Mirvetuximab Soravtansine. Simultaneously applying the alternating electric field and administering the antibody-drug conjugate comprising Mirvetuximab Soravtansine can include applying the alternating electric field while administering the antibody-drug conjugate comprising Mirvetuximab Soravtansine. Preferably, the frequency of the alternating electric field is 200 kHz, and the strength is 1.7V/cm; the recommended dose of the antibody-drug conjugate comprising Mirvetuximab Soravtansine is 6 mg/kg once every 3 weeks for intravenous infusion (every 21 days is a treatment course) until disease progresses or unacceptable toxicity occurs. It should be noted that the recommended dose of antibody-drug conjugates comprising Mirvetuximab Soravtansine also needs to be determined based on the adjusted ideal body weight (AIBW).

In some embodiments, the antibody-drug conjugate comprising Mirvetuximab Soravtansine can be Elahere®.

Elahere® (Mirvetuximab Soravtansine-gynx; IMGN-853) is a folate receptor (FRα) targeted antibody-drug conjugate developed by ImmunoGen. It consists of three parts: a humanized anti-FRα monoclonal antibody, a small molecule toxic drug DM4, and a linker sulfo-SPDB. When Elahere® binds to FRα, FRα can transfer the drug into the inside of the cell. After the linker cleavage, DM4 inhibits microtubule protein aggregation by binding to the microtubule proteins, thereby causing cell cycle arrest and inducing apoptosis of tumor cells. On Nov. 14, 2022, Elahere® was granted an accelerated approval in the U.S. for the treatment of adult patients with FRα-positive platinum-resistant ovarian epithelial cancer, fallopian tube cancer, or primary peritoneal cancer who have received 1-3 lines of systemic treatment regimens. Elahere® has thus become the first antibody-drug conjugate approved by the U.S. FDA for platinum-resistant ovarian cancer.

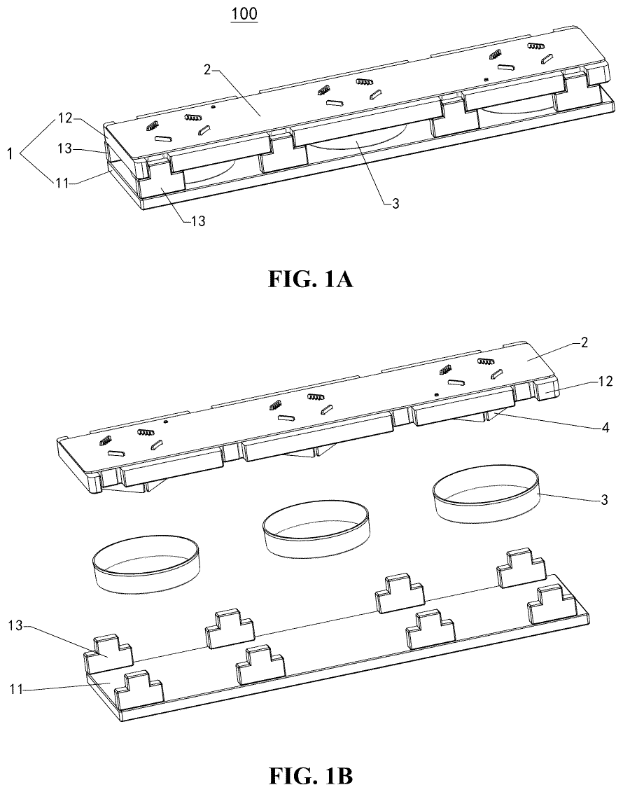

Referring to FIGS. 1A and 1B, this application also provides a TTFields in vitro cell experimental system, which comprises a tumor electric field therapy device (not shown in figure) and a TTFields cell experimental device 100 electrically connected to the tumor electric field therapy device (not shown in figure) for applying an alternating electric signal to experimental cells. The TTFields cell experimental device 100 comprises a main body 1, a circuit board 2, multiple culture dishes 3, and multiple pairs of electrode plates 4. The main body 1 comprises a base plate 11, a top plate 12, and multiple support plates 13 supported between the base plate 11 and the top plate 12. The circuit board 2 is flatly fixed on the top surface of the top plate 12. The culture dishes 3 are horizontally placed on the base plate 11 and are located between the base plate 11 and the top plate 12. The electrode plates 4 are placed between the culture dishes 3 and the top plate 12 through the bottom thereof being inserted into the culture dishes 3 and the top thereof passing upward through the top plate 12 and being plugged into and connected to the circuit board 2. The culture dishes 3 contain culture medium, and the types of which can be the same or different, depending on the experimental requirements. An electric field generator (not shown in figure) of the tumor electric field therapy device (not shown in figure) is connected to the circuit board 2, and the alternating electric signal generated by the electric field generator (not shown in figure) of the tumor electric field therapy device (not shown in figure) applies TTFields to tumor cells in the culture medium through the circuit board 2 and the electrode plates 4 to study the effects of factors such as field frequency, field strength, direction, and exposure time of TTFields on the growth of tumor cells. Said TTFields in vitro cell experimental system can refer to CN218561476U-TTFields cell experimental device, the disclosure of this utility model is incorporated by reference in its entirety herein.

In this application, all cell experiments are conducted in the TTFields in vitro cell experimental system. Since the higher the output voltage of the electric field therapy device (not shown in figure), the stronger the alternating electric field strength generated by it, thereby producing more heat, in order to ensure cell survival, the electrode plates 4 are equipped with a thermistor (not shown in figure) that can record real-time culture medium temperature data. During the experiment, a specific incubator environmental temperature of 33-34° C. is set according to the voltage level of the electric field therapy device (not shown in figure), thereby controlling the temperature inside the culture medium within the range of 36.5-37.5° C.

In the following examples, the antibody-drug conjugate Enhertu® was purchased from Selleck Company with a catalog number of E0200 and a batch number of E020001; Tivdak® was purchased from Selleck Company with a catalog number of D4051 and a batch number of D405101; Trodelvy® was purchased from Selleck Company with a catalog number of E2841 and a batch number of E284101; and Elahere® was purchased from MedChemExpress Company with a catalog number of HY-132258.

### Example 1—Inhibition of the Growth of NCI-N87 Gastric Cancer Cells by Enhertu® in Combination with TTFields

### 1.1 Cell Culture

The growth medium for NCI-N87 cells consists of 10% fetal bovine serum, 1% Penicillin-Streptomycin Solution, and 90% RPMI-1640 medium (Roswell Park Memorial Institute-1640). All cells were placed in a constant-temperature cell culture incubator at 37° C. for incubation with 95% air and 5% CO₂ under a saturated humidity condition. NCI-N87 cells from the incubator were transferred to culture dishes, digested with trypsin containing 0.25% EDTAand then seeded on 20 mm round coverslips in 12-well plates. The cells were further incubated in a constant-temperature cell culture incubator at 37° C. with 5% CO₂ under a saturated humidity condition for 72 hours.

### 1.2 Selection of Optimal Frequency for TTFields

12 coverslips of NCI-N87 under good cell condition were selected. 3 coverslips per group were placed in 4 culture dishes respectively. After adding 15 ml of culture medium to the dishes, three dishes were placed in three TTFields cell experimental devices. The TTFields in vitro cell experimental system was used to apply an alternating electric field of different frequencies (100 kHz, 150 kHz, and 200 kHz) and a strength of 1.6 V/cm to the cell samples in the three dishes. Subsequently, the three TTFields cell experimental devices cach containing one dish and the other dish without applying TTFields were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 96 hours.

### 1.3 Cytotoxicity Test and Conclusion

After 96 hours of treatment for NCI-N87 cells, the coverslips from each group were taken out, and the NCI-N87 cells were digested with trypsin containing 0.25% EDTA to form a single-cell suspension. The number of NCI-N87 cells in each group was detected through a handheld cell counter Scepter 3.0, and the cytotoxic effect was determined based on the number of NCI-N87 cells.

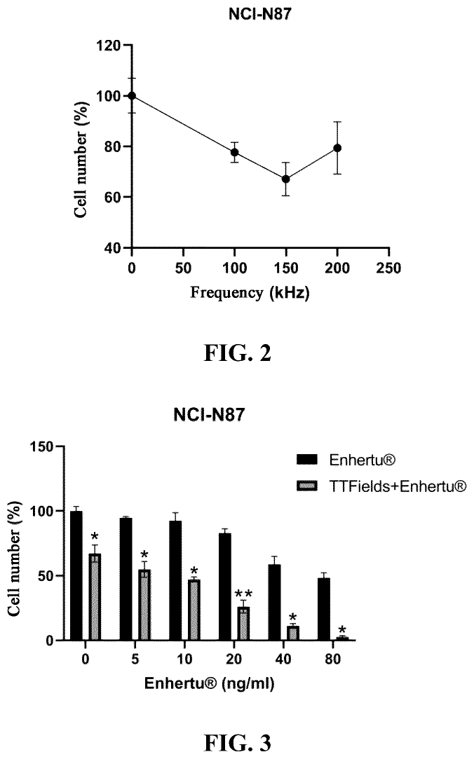

Referring to FIG. 2, the cell counting experiment results show that the number of cells in the TTFields groups with different frequencies has decreased compared to the group without applying TTFields, with the most significant reduction in the TTFields group with a frequency of 150 kHz. Therefore, the TTFields with a frequency of 150 kHz have the optimal inhibitory effect on the growth of NCI-N87 cells, and the TTFields with a frequency of 150 kHz in combination with Enhertu® against NCI-N87 cells were eventually selected for the next experiment of inhibiting the growth of the cancer cells.

### 1.4 Combination Treatment of Enhertu® and TTFields

36 coverslips of NCI-N87 under good cell condition were selected. 3 coverslips per group were placed in 12 culture dishes respectively. The 12 dishes were divided into two groups. One group was treated with Enhertu® alone and included 6 dishes cultured with different concentrations of Enhertu® (0 ng/ml, 5 ng/ml, 10 ng/ml, 20 ng/ml, 40 ng/ml, and 80 ng/ml) in the medium. The other group was treated with Enhertu® and simultaneously applied with TTFields, and included 6 dishes cultured with different concentrations of Enhertu® (0 ng/ml, 5 ng/ml, 10 ng/ml, 20 ng/ml, 40 ng/ml, and 80 ng/ml) in the medium. The total 6 dishes of this group were placed in the TTFields cell experimental device, and the TTFields in vitro cell experimental system was used to apply an alternating electric field with a frequency of 150 kHz and a strength of 1.6 V/cm to the cell samples in the dishes. Subsequently, the TTFields cell experimental devices were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 96 hours.

### 1.5 Cytotoxicity Detection and Conclusion

After 96 hours of treatment for NCI-N87 cells, the coverslips from each group were taken out, and the NCI-N87 cells were digested with trypsin containing 0.25% EDTA to form a single-cell suspension. The number of NCI-N87 cells in each group was detected through a handheld cell counter Scepter 3.0, and the cytotoxic effect was determined based on the number of NCI-N87 cells.

As shown in FIG. 3, the cell counting experiment results show that the cytotoxic effect of the combination of applying TTFields and administering Enhertu® on NCI-N87 cells was significantly higher than that of administering Enhertu® alone or applying TTFields alone (two-way ANOVA, followed by multiple comparisons, *P<0.05; **P<0.01).

### 1.6 Cell Clone Formation and Conclusion

After the cell counted, a small number of NCI-N87 cells selected from the groups treated with three concentrations of Enhertu® (20 ng/ml, 40 ng/ml, and 80 ng/ml) were respectively seeded in 6-well plates, and each 6-well plate had 8000 NCI-N87 cells in each well thereof. The cells were further cultured until the majority of the individual clones have more than 50 cells. The medium was changed every 3 days during the culture and the cell condition was observed. After the clones were completed, the cells were washed once with PBS, and fixed with 4% paraformaldehyde for 30 minutes, and then washed once with PBS, and then stained with 1% crystal violet for 10 minutes per well. The cells were washed several times with PBS, dried, photographed, and counted. The clone formation rate was equal to the ratio of the number of clones to the number of seeded cells, and the clonogenic effect was determined by the clone formation rate.

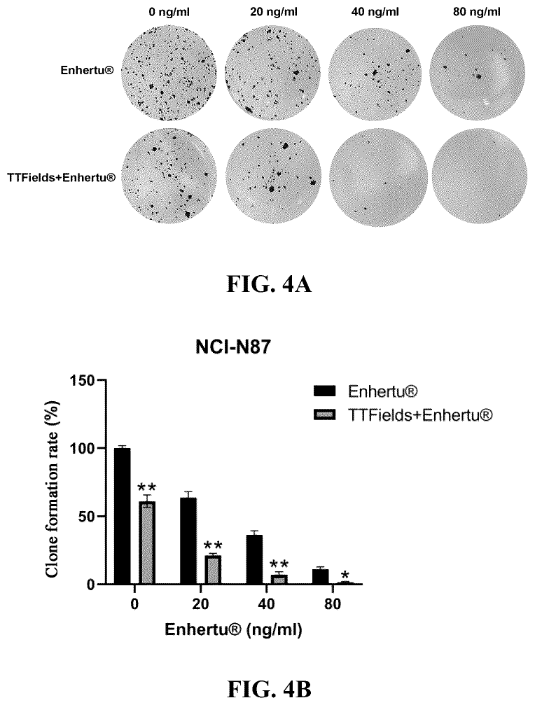

As shown in FIGS. 4A and 4B, the clonogenic effect results show that the number of colonies of cells treated by the combination of TTFields and Enhertu® is significantly reduced compared to that of administering Enhertu® alone or applying TTFields alone (two-way ANOVA, followed by multiple comparisons, *P<0.05; **P<0.01).

### Example 2—Inhibition of the Growth of CaSki Cervical Cancer Cells by Tivdak® in Combination with TTFields

### 2.1 Cell Culture

The growth medium for CaSki cells consists of 10% fetal bovine serum, 1% Penicillin-Streptomycin Solution, and 90% RPMI-1640 medium (Roswell Park Memorial Institute-1640). All cells were placed in a constant-temperature cell culture incubator at 37° C. for incubation with 95% air and 5% CO₂ under a saturated humidity condition. CaSki cells from the incubator were transferred to culture dishes, digested with trypsin containing 0.25% EDTA and then seeded on 20 mm round coverslips in 12-well plates. The cells were further incubated in a constant-temperature cell culture incubator at 37° C. with 5% CO₂ under a saturated humidity condition for 72 hours.

### 2.2 Selection of Optimal Frequency for TTFields

12 coverslips of CaSki under good cell condition were selected. 3 coverslips per group were placed in 4 culture dishes respectively. After adding 15 ml of culture medium to the dishes, three dishes were placed in three TTFields cell experimental devices. The TTFields in vitro cell experimental system was used to apply an alternating electric field of different frequencies (150 kHz, 200 kHz, and 250 kHz) and a strength of 1.7V/cm to the cell samples in the three dishes. Subsequently, the three TTFields cell experimental devices each containing one dish and the other dish without applying TTFields were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 96 hours.

### 2.3 Cytotoxicity Test and Conclusion

After 96 hours of treatment for CaSki cells, the coverslips from each group were taken out, and the CaSki cells were digested with trypsin containing 0.25% EDTA to form a single-cell suspension. The number of CaSki cells in each group was detected through a handheld cell counter Scepter 3.0, and the cytotoxic effect was determined according to the number of CaSki cells.

The cell counting experiment results show that the number of cells in the TTFields groups with different frequencies has decreased compared to the group without applying TTFields, with the most significant reduction in the TTFields group with a frequency of 200 kHz. Therefore, the TTFields with a frequency of 200 kHz have the optimal inhibitory effect on the growth of CaSki cells, and the TTFields with a frequency of 200 kHz in combination with Tivdak® against CaSki cells were eventually selected for the next experiment of inhibiting the growth of the cancer cells.

### 2.4 Combination Treatment of Tivdak® and TTFields

36 coverslips of CaSki under good cell condition were selected. 3 coverslips per group were placed in 12 culture dishes respectively. The 12 dishes were divided into two groups. One group was treated with Tivdak® alone, and included 6 dishes cultured with different concentrations of Tivdak® (0 ng/ml, 1 ng/ml, 2 ng/ml, 4 ng/ml, 8 ng/ml, and 16 ng/ml) in the medium. The other group was treated with Tivdak® and simultaneously applied with TTFields, and included 6 dishes cultured with different concentrations of Tivdak® (0 ng/ml, 1 ng/ml, 2 ng/ml, 4 ng/ml, 8 ng/ml, and 16 ng/ml) in the medium. The total 6 dishes of this group were placed in the TTFields cell experimental device, and the TTFields in vitro cell experimental system was used to apply an alternating electric field with a frequency of 200 kHz and a strength of 1.7V/cm to the cell samples in the dishes. Subsequently, the TTFields cell experimental devices were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 96 hours.

### 2.5 Cytotoxicity Detection and Conclusion

After 96 hours of treatment for CaSki cells, the coverslips from each group were taken out, and the CaSki cells were digested with trypsin containing 0.25% EDTA to form a single-cell suspension. The number of CaSki cells in each group was detected through a handheld cell counter Scepter 3.0, and the cytotoxic effect was determined based on the number of CaSki cells.

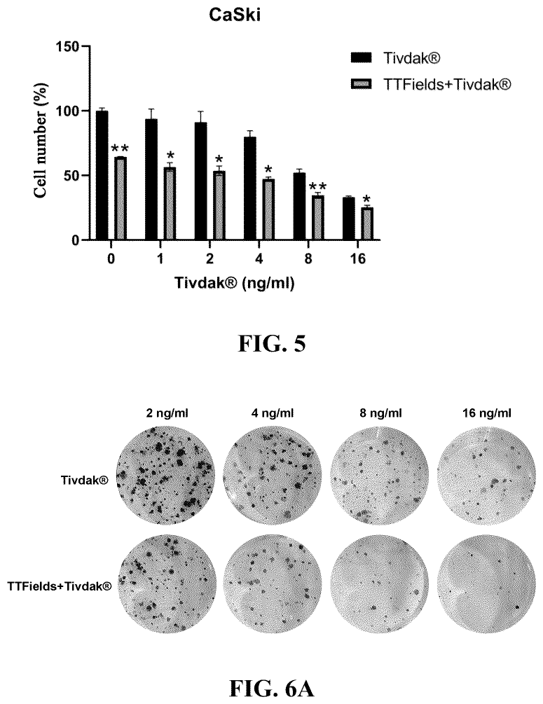

Referring to FIG. 5, the cell counting experiment results show that the cytotoxic effect of the combination of applying TTFields and administering Tivdak® on CaSki cells was significantly higher than that of administering Tivdak® alone or applying TTFields alone (two-way ANOVA, followed by multiple comparisons, *P<0.05; **P<0.01).

### 2.6 Cell Clone Formation and Conclusion

After the cell counted, a small number of CaSki cells from the groups treated with three Tivdak® concentrations (4 ng/ml, 8 ng/ml, and 16 ng/ml) were respectively seeded in 6-well plates, and each 6-well plat had 3000 CaSki cells in each well thereof. The cells were further cultured until the majority of the individual clones have more than 50 cells. The medium was changed every 3 days during the culture and the cell condition was observed. After the clones were completed, the cells were washed once with PBS, and fixed with 4% paraformaldehyde for 30 minutes, and then washed once with PBS, and then stained with 1% crystal violet for 10 minutes per well. The cells were washed several times with PBS, dried, photographed, and counted. The clone formation rate was determined as the ratio of the number of clones to the number of seeded cells, and the clonogenic effect was determined by the clone formation rate.

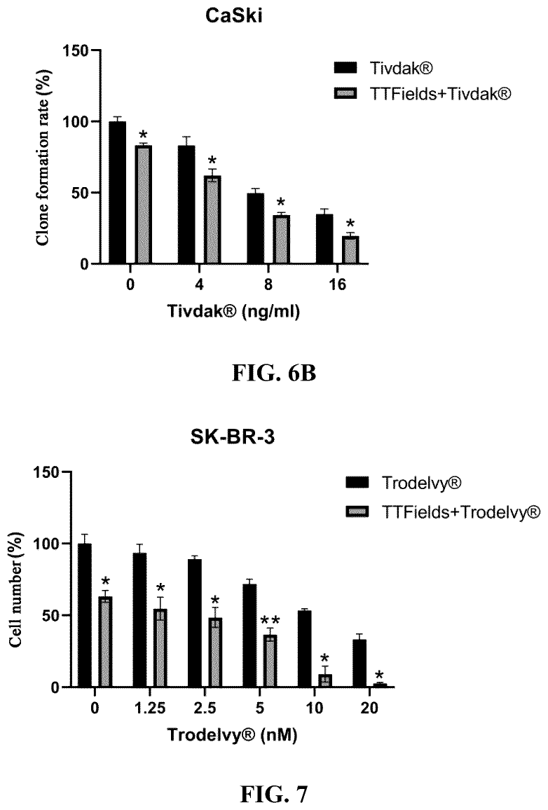

Referring to FIGS. 6A and 6B, the clonogenic effect results show that the number of colonies of cells treated by the combination of TTFields and Tivdak® is significantly reduced compared to that of administering Tivdak® alone or applying TTFields alone (two-way ANOVA, followed by multiple comparisons, *P<0.05).

### Example 3—Inhibition of the Growth of SK-BR-3 Breast Cancer Cells by Trodelvy® in Combination with TTFields

### 3.1 Cell Culture

The growth medium for SK-BR-3 cells consists of 10% fetal bovine serum, 1% Penicillin-Streptomycin Solution, and 90% High-glucose DMEM (Dulbecco's Modified Eagle Medium). All cells were placed in a constant-temperature cell culture incubator at 37° C. for incubation with 95% air and 5% CO₂ under a saturated humidity condition. SK-BR-3 cells from the incubator were transferred to culture dishes, digested with trypsin containing 0.25% EDTA and then seeded on 20 mm round coverslips in 12-well plates. The cells were further incubated in a constant-temperature cell culture incubator at 37° C. with 5% CO₂ under a saturated humidity condition for 24 hours.

### 3.2 Selection of Optimal Frequency for TTFields

12 coverslips of SK-BR-3 under good cell condition were selected. 3 coverslips per group were placed in 4 culture dishes respectively. After adding 15 ml of culture medium to the dishes, three dishes were placed in three TTFields cell experimental devices. The TTFields in vitro cell experimental system was used to apply an alternating electric field of different frequencies (100 kHz, 150 kHz, and 200 kHz) and a strength of 1.7V/cm to the cell samples in the three dishes. Subsequently, the three TTFields cell experimental devices each containing one dish and the other dish without applying TTFields were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 72 hours.

### 3.3 Cytotoxicity Test and Conclusion

After 72 hours of treatment for SK-BR-3 cells, the coverslips from each group were taken out, and the SK-BR-3 cells were digested with trypsin containing 0.25% EDTA to form a single-cell suspension. The number of SK-BR-3 cells in each group was detected through a handheld cell counter Scepter 3.0, and the cytotoxic effect was determined based on the number of SK-BR-3 cells counted.

The cell counting experiment results show that the number of cells in the TTFields groups with different frequencies has decreased compared to the group without applying TTFields, with the most significant reduction in the TTFields group with a frequency of 150 kHz. Therefore, the TTFields with a frequency of 150 kHz have the optimal inhibitory effect on the growth of SK-BR-3 cells, and the TTFields with a frequency of 150 kHz in combination with Trodelvy® against SK-BR-3 cells were eventually selected for the next experiment of inhibiting the growth of the cancer cells.

### 3.4 Combination Treatment of Trodelvy® and TTFields

36 coverslips of SK-BR-3 under good cell condition were selected. 3 coverslips per group were placed in 12 culture dishes respectively. The 12 dishes were divided into two groups. One group was treated with Trodelvy® alone, and included 6 dishes cultured with different concentrations of Trodelvy® (0 nM, 1.25 nM, 2.5 nM, 5 nM, 10 nM, and 20 nM) in the medium. The other group was treated with Trodelvy® and simultaneously applied with TTFields, and also included 6 dishes cultured with different concentrations of Trodelvy® (OnM, 1.25 nM, 2.5 nM, 5 nM, 10 nM, and 20 nM) in the medium. The total 6 dishes of this group were placed in the TTFields cell experimental device, and the TTFields in vitro cell experimental system was used to apply an alternating electric field with a frequency of 150 kHz and a strength of 1.7V/cm to the cell samples in the dishes. Subsequently, the TTFields cell experimental devices were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 72 hours.

### 3.5 Cytotoxicity Detection and Conclusion

After 72 hours of treatment for SK-BR-3 cells, the coverslips from each group were taken out, and the SK-BR-3 cells were digested with trypsin containing 0.25% EDTA to form a single-cell suspension. The number of SK-BR-3 cells in each group was detected through a handheld cell counter Scepter 3.0, and the cytotoxic effect was determined based on the number of SK-BR-3 cells.

Referring to FIG. 7, the cell counting experiment results show that the cytotoxic effect of the combination of applying TTFields and administering Trodelvy® on SK-BR-3 cells was significantly higher than that of administering Trodelvy® alone or applying TTFields alone (two-way ANOVA, followed by multiple comparisons, *P<0.05; **P<0.01).

### 3.6 Cell Clone Formation and Conclusion

After the cell counted, a small number of SK-BR-3 cells from the groups treated with three Trodelvy® concentrations (5 nM, 10 nM, and 20 nM) were respectively seeded in 6-well plates, and each 6-well plate had 200 SK-BR-3 cells in each well thereof. The cells were further cultured until the majority of the individual clones have more than 50 cells. The medium was changed every 3 days during the culture and the cell condition was observed. After the clones were completed, the cells were washed once with PBS, and fixed with 4% paraformaldehyde for 30 minutes, and then washed once with PBS, and then stained with 1% crystal violet for 10 minutes per well. The cells were washed several times with PBS, dried, photographed, and counted. The clone formation rate was determined as the ratio of the number of clones to the number of seeded cells, and the clonogenic effect was determined by the clone formation rate.

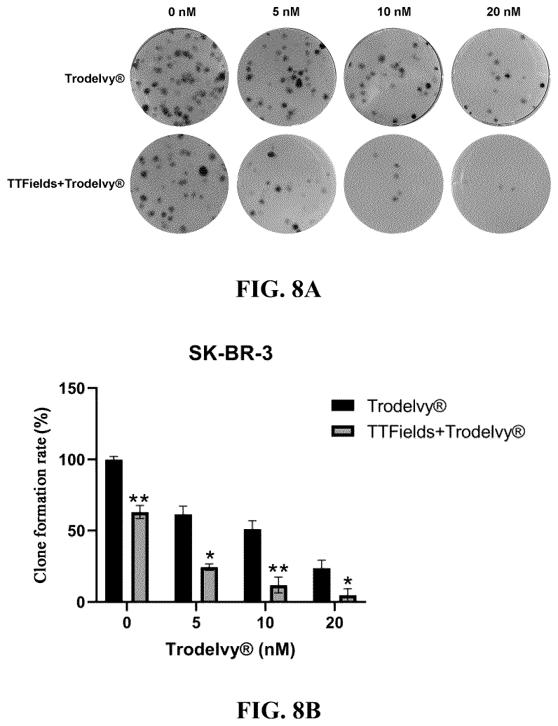

Referring to FIGS. 8A and 8B, the clonogenic effect results show that the number of colonies of cells treated with the combination of TTFields and Trodelvy® is significantly reduced compared to cells treated with administering Trodelvy® alone or applying TTFields alone (two-way ANOVA, followed by multiple comparisons, *P<0.05; **P<0.01).

### Example 4—Inhibition of the Growth of SK-OV-3 Ovarian Cancer Cells by Elahere® in Combination with TTFields

### 4.1 Cell Culture

The growth medium for SK-OV-3 cells consists of 10% fetal bovine serum, 1% Penicillin-Streptomycin Solution, and 90% High-glucose DMEM (Dulbecco's Modified Eagle Medium). All cells were placed in a constant-temperature cell culture incubator at 37° C. for incubation with 95% air and 5% CO₂ under a saturated humidity condition. SK-OV-3 cells from the incubator were transferred to culture dishes, digested with trypsin containing 0.25% EDTA and then seeded on 20 mm round coverslips in 12-well plates. The cells were further incubated in a constant-temperature cell culture incubator at 37° C. with 5% CO₂ under a saturated humidity condition for 24 hours.

### 4.2 Selection of Optimal Frequency for TTFields

12 coverslips of SK-OV-3 under good cell condition were selected. 3 coverslips per group were placed in 4 culture dishes respectively. After adding 15 ml of culture medium to the dishes, three dishes were placed in three TTFields cell experimental devices. The TTFields in vitro cell experimental system was used to apply an alternating electric field of different frequencies (150 kHz, 200 kHz, and 250 kHz) and a strength of 1.7V/cm to the cell samples in the three dishes. Subsequently, the three TTFields cell experimental devices each containing one dish and the other dish without applying TTFields were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 72 hours.

### 4.3 Cytotoxicity Test and Conclusion

After 72 hours of treatment for SK-OV-3 cells, the coverslips from each group were taken out, and the SK-OV-3 cells were digested with trypsin containing 0.25% EDTA to form a single-cell suspension. The number of SK-OV-3 cells in each group was detected through a handheld cell counter Scepter 3.0, and the cytotoxic effect was determined according to the number of SK-OV-3 cells.

The cell counting experiment results show that the number of cells in the TTFields groups with different frequencies has decreased compared to the group without applying TTFields, with the most significant reduction in the TTFields group with a frequency of 200 kHz. Therefore, the TTFields with a frequency of 200 kHz have the optimal inhibitory effect on the growth of SK-OV-3 cells, and the TTFields with a frequency of 200 kHz in combination with Elahere® against SK-OV-3 cells were eventually selected for the next experiment of inhibiting the growth of the cancer cells.

### 4.4 Combination Treatment of Elahere® and TTFields

36 coverslips of SK-OV-3 under good cell condition were selected. 3 coverslips per group were placed in 12 culture dishes respectively. The 12 dishes were divided into two groups. One group was treated with Elahere® alone, and included 6 dishes cultured with different concentrations of Elahere® (0 nM, 0.5 nM, 1 nM, 2 nM, 4 nM, and 8 nM) in the medium. The other group was treated with Elahere® and simultaneously applied with TTFields, and also included 6 dishes cultured with different concentrations of Elahere® (0 nM, 0.5 nM, 1 nM, 2 nM, 4 nM, and 8 nM) in the medium. The total 6 dishes of this group were placed in the TTFields cell experimental device, and the TTFields in vitro cell experimental system was used to apply an alternating electric field with a frequency of 200 kHz and a strength of 1.7V/cm to the cell samples in the dishes. Subsequently, the TTFields cell experimental devices were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 72 hours.

### 4.5 Cytotoxicity Detection and Conclusion

After 72 hours of treatment for SK-OV-3 cells, the coverslips from each group were taken out, and the SK-OV-3 cells were digested with trypsin containing 0.25% EDTA to form a single-cell suspension. The number of SK-OV-3 cells in each group was detected through a handheld cell counter Scepter 3.0, and the cytotoxic effect was determined by the number of SK-OV-3 cells counted.

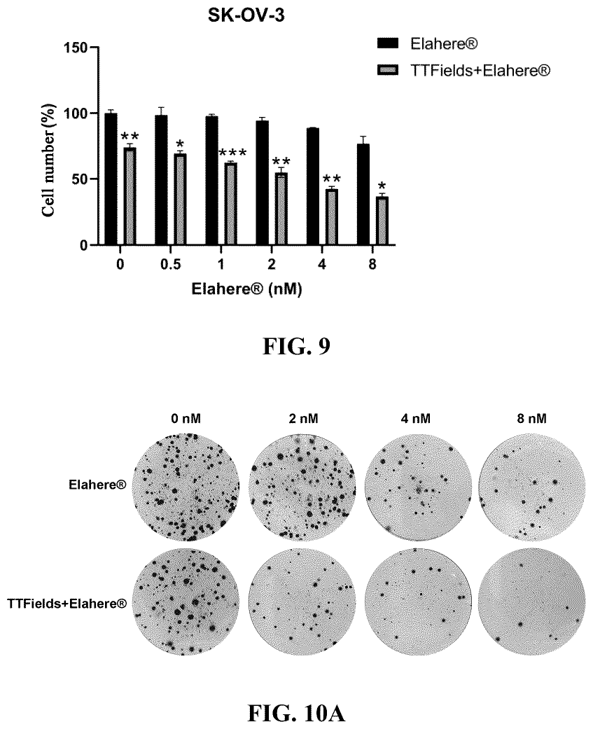

Referring to FIG. 9, the cell counting experiment results show that the cytotoxic effect of the combination of applying TTFields and administering Elahere® on SK-OV-3 cells was significantly higher than that of administering Elahere® alone or applying TTFields alone (two-way ANOVA, followed by multiple comparisons, *P<0.05; **P<0.01; ***P<0.001).

### 4.6 Cell Clone Formation and Conclusion

After the cell counted, a small number of SK-OV-3 cells from the groups treated with three Elahere® concentrations (5 nM, 10 nM, and 20 nM) were respectively seeded in 6-well plates, and each 6-well plate had 400 SK-OV-3 cells in each well thereof. The cells were further cultured until the majority of the individual clones have more than 50 cells. The medium was changed every 3 days during the culture and the cell condition was observed. After the clones were completed, the cells were washed once with PBS, and fixed with 4% paraformaldehyde for 30 minutes, and then washed once with PBS, and then stained with 1% crystal violet for 10 minutes per well. The cells were washed several times with PBS, dried, photographed, and counted. The clone formation rate was determined as the ratio of the number of clones to the number of seeded cells, and the clonogenic effect was determined by the clone formation rate.

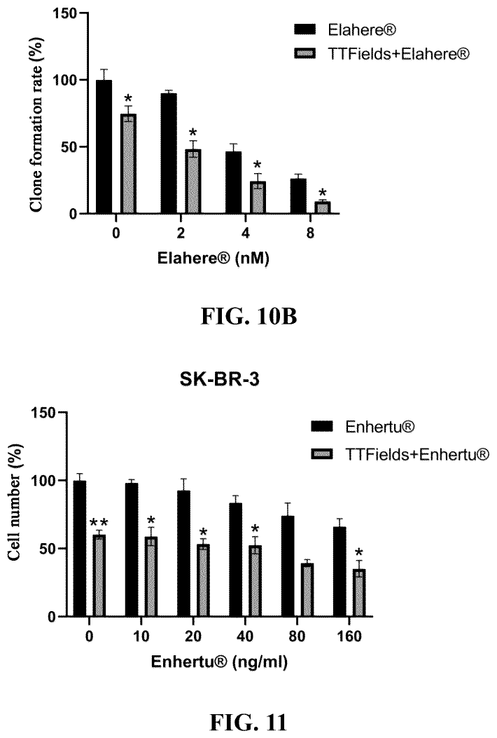

Referring to FIGS. 10A and 10B, the clonogenic effect results show that the number of colonies of cells treated with the combination of TTFields and Elahere® is significantly reduced compared to that of administering Elahere® alone or applying TTFields alone (two-way ANOVA, followed by multiple comparisons, *P<0.05).

### Example 5—Inhibition of the Growth of SK-BR-3 Breast Cancer Cells by Enhertu® in Combination with TTFields

### 5.1 Cell Culture

The growth medium for SK-BR-3 cells consists of 10% fetal bovine serum, 1% Penicillin-Streptomycin Solution, and 90% High-glucose DMEM (Dulbecco's Modified Eagle Medium). All cells were placed in a constant-temperature cell culture incubator at 37° C. for incubation with 95% air and 5% CO₂ under a saturated humidity condition. SK-BR-3 cells from the incubator were transferred to culture dishes, digested with trypsin containing 0.25% EDTA and then seeded on 20 mm round coverslips in 12-well plates. The cells were further incubated in a constant-temperature cell culture incubator at 37° C. with 5% CO₂ under a saturated humidity condition for 24 hours.

### 5.2 Selection of Optimal Frequency for TTFields

12 coverslips of SK-BR-3 under good cell condition were selected. 3 coverslips per group were placed in 4 culture dishes respectively. After adding 15 ml of culture medium to the dishes, three dishes were placed in three TTFields cell experimental devices. The TTFields in vitro cell experimental system was used to apply an alternating electric field of different frequencies (100 kHz, 150 kHz, and 200 kHz) and a strength of 1.7V/cm to the cell samples in the three dishes. Subsequently, the three TTFields cell experimental devices cach containing one dish and the other dish without applying TTFields were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 72 hours.

### 5.3 Cytotoxicity Test and Conclusion

After 72 hours of treatment for SK-BR-3 cells, the coverslips from each group were taken out, and the SK-BR-3 cells were digested with trypsin containing 0.25% EDTA to form a single-cell suspension. The number of SK-BR-3 cells in each group was detected through a handheld cell counter Scepter 3.0, and the cytotoxic effect was determined by the number of SK-BR-3 cells counted.

The cell counting experiment results show that the number of cells in the TTFields groups with different frequencies has decreased compared to the group without applying TTFields, with the most significant reduction in the TTFields group with a frequency of 150 kHz. Therefore, the TTFields with a frequency of 150 kHz have the optimal inhibitory effect on the growth of SK-BR-3 cells, and the TTFields with a frequency of 150 kHz in combination with Enhertu® against SK-BR-3 cells were eventually selected for the next experiment of inhibiting the growth of the cancer cells.

### 5.4 Combination Treatment of Enhertu® and TTFields

36 coverslips of SK-BR-3 under good cell condition were selected. 3 coverslips per group were placed in 12 culture dishes respectively. The 12 dishes were divided into two groups. One group was treated with Enhertu® alone, and included 6 dishes cultured with different concentrations of Enhertu® (0 ng/ml, 10 ng/ml, 20 ng/ml, 40 ng/ml, 80 ng/ml, and 160 ng/ml) in the medium. The other group was treated with Enhertu® and simultaneously applied with TTFields, and also included 6 dishes cultured with different concentrations of Enhertu® (0 ng/ml, 10 ng/ml, 20 ng/ml, 40 ng/ml, 80 ng/ml, and 160 ng/ml) in the medium. The 6 dishes of this group were placed in the TTFields cell experimental device, and the TTFields in vitro cell experimental system was used to apply an alternating electric field with a frequency of 150 kHz and a strength of 1.7V/cm to the cell samples in the dishes. Subsequently, the TTFields cell experimental devices were placed in a constant-temperature cell culture incubator at 33-34° C. (adjusted according to the electric field strength to control the culture medium temperature within the range of 36.5-37.5° C.) for incubation with 5% CO₂ under a saturated humidity condition for 72 hours. 5.5 Cytotoxicity Detection and Conclusion

After 72 hours of treatment for SK-BR-3 cells, the coverslips from each group were taken out, and the SK-BR-3 cells were digested with trypsin containing 0.25% EDTA to form a single-cell suspension. The number of SK-BR-3 cells in each group was detected through a handheld cell counter Scepter 3.0, and the cytotoxic effect was determined by the number of SK-BR-3 cells counted.

Referring to FIG. 11, the cell counting experiment results show that the cytotoxic effect of the combination of applying TTFields and administering Enhertu® on SK-BR-3 cells was significantly higher than that of administering Enhertu® alone or applying TTFields alone (two-way ANOVA, followed by multiple comparisons, *P<0.05; **P<0.01).

### Example 6—Inhibition of the Growth of NCI-H1781 Non-Small Cell Lung Cancer Cells by Enhertu® in Combination with TTFields

### 6.1 Cell Culture

The growth medium for NCI-H1781 cells consists of 10% fetal bovine serum, 1% Penicillin-Streptomycin Solution, and 90% RPMI-1640 (Roswell Park Memorial Institute-1640). All cells were placed in a constant-temperature cell culture incubator at 37° C. for incubation with 95% air and 5% CO₂ under a saturated humidity condition. NCI-H1781 cells from the incubator were transferred to culture dishes, digested with trypsin containing 0.25% EDTA and then seeded on 20 mm round coverslips in 12-well plates. The cells were further incubated in a constant-temperature cell culture incubator at 37° C. with 5% CO₂ under a saturated humidity condition for 96 hours.

### 6.2 Selection of Optimal Frequency for TTFields

12 coverslips of NCI-H1781 under good cell condition were selected. 3 coverslips per group were placed in 4 culture dishes respectively. After adding 15 ml of culture medium to the dishes, three dishes were placed in three TTFields cell experimental devices. The TTFields in vitro cell experimental system was used to apply an alternating electric field of different frequencies (100 kHz, 150 kHz, and 200 kHz) and a strength of 1.8V/cm to the cell samples in the three dishes. Subsequently, the three TTFields cell experimental devices each containing one dish were placed in a constant-temperature cell culture incubator at 33-34° C. (adjusted according to the electric field strength to control the culture medium temperature within the range of 36.5-37.5° C.) and 5% CO₂. One dish without applying TTFields was placed in a constant-temperature cell culture incubator at 36.5-37.5° C. and 5% CO₂. Both of them were incubated under a saturated humidity condition for 96 hours.

### 6.3 Cytotoxicity Test and Conclusion

After 96 hours of treatment for NCI-H1781 cells, the coverslips from each group were taken out, and the NCI-H1781 cells were digested with trypsin containing 0.25% EDTA to form a single-cell suspension. The number of NCI-H1781 cells in each group was detected through a handheld cell counter Scepter 3.0, and the cytotoxic effect was determined by the number of NCI-H1781 cells counted.

The cell counting experiment results show that the number of cells in the TTFields groups with different frequencies has decreased compared to the group without applying TTFields, with the most significant reduction in the TTFields group with a frequency of 150 kHz. Therefore, the TTFields with a frequency of 150 kHz have the optimal inhibitory effect on the growth of NCI-H1781 cells, and the TTFields with a frequency of 150 kHz in combination with Enhertu® against NCI-H1781 cells were eventually selected for the next experiment of inhibiting the growth of the cancer cells.

### 6.4 Combination Treatment of Enhertu® and TTFields

36 coverslips of NCI-H1781 under good cell condition were selected. 3 coverslips per group were placed in 12 culture dishes respectively. The 12 dishes were divided into two groups. One group was treated with Enhertu® alone, and included 6 dishes cultured with different concentrations of Enhertu® (0 ng/ml, 2.5 ng/ml, 5 ng/ml, 10 ng/ml, 20 ng/ml, and 40 ng/ml) in the medium. The other group was treated with Enhertu® and simultaneously applied with TTFields, and also included 6 dishes cultured with different concentrations of Enhertu® (0 ng/ml, 2.5 ng/ml, 5 ng/ml, 10 ng/ml, 20 ng/ml, and 40 ng/ml) in the medium. The total 6 dishes of this group were placed in the TTFields cell experimental device, and the TTFields in vitro cell experimental system was used to apply an alternating electric field with a frequency of 150 kHz and a strength of 1.8V/cm to the cell samples in the dishes. Subsequently, the TTFields cell experimental devices were placed in a constant-temperature cell culture incubator at 33-34° C. (adjusted according to the electric field strength to control the culture medium temperature within the range of 36.5-37.5° C.) and 5% CO₂. The 6 dishes administered with Enhertu® alone and without applying TTFields were placed in a constant-temperature cell culture incubator at 36.5-37.5° C. and 5% CO₂. Both of them were incubated under a saturated humidity condition for 96 hours.

### 6.5 Cytotoxicity Detection and Conclusion

After 96 hours of treatment for NCI-H1781 cells, the coverslips from each group were taken out, and the NCI-H1781 cells were digested with trypsin containing 0.25% EDTA to form a single-cell suspension. The number of NCI-H1781 cells in each group was detected by a handheld cell counter Scepter 3.0, and the cytotoxic effect was determined by the number of NCI-H1781 cells counted.

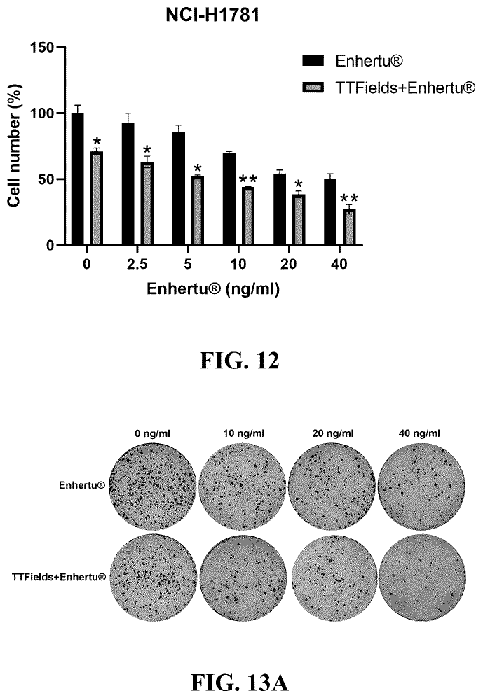

Referring to FIG. 12, the cell counting experiment results show that the cytotoxic effect of the combination of applying TTFields and administering Enhertu® on NCI-H1781 cells was significantly higher than that of administering E nhertu® alone or applying TTFields alone (two-way ANOVA, followed by multiple comparisons, *P<0.05; **P<0.01).

### 6.6 Cell Clone Formation and Conclusion

After the cell counted, a small number of NCI-H1781 cells from the three Enhertu® concentration groups (10 ng/ml, 20 ng/ml, and 40 ng/ml) were respectivly seeded in 6-well plates, and each 6-well plate had 10000 NCI-H1781 cells in each well thereof. The cells were further cultured until the majority of the individual clones have more than 50 cells. The medium was changed every 3 days during the culture and the cell condition was observed. After the clones were completed, the cells were washed once with PBS, fixed with 4% paraformaldehyde for 30 minutes, washed once with PBS, and then stained with 1% crystal violet for 10 minutes per well. The cells were washed several times with PBS, dried, photographed, and counted. The clone formation rate was determined as the ratio of the number of clones to the number of seeded cells, and the clonogenic effect was determined by the clone formation rate.

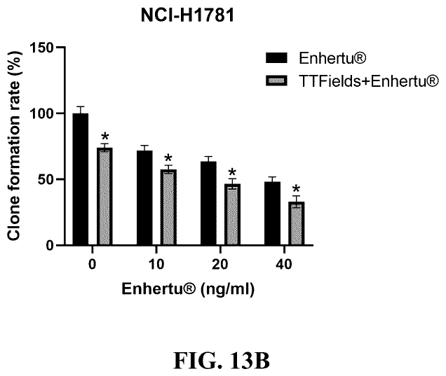

Referring to FIGS. 13A and 13B, the clonogenic effect results show that the number of colonies of cells treated with the combination of TTFields and Enhertu® is significantly reduced compared to that of administering Enhertu® alone or applying TTFields alone (two-way ANOVA, followed by multiple comparisons, *P<0.05; **P<0.01).

### Conclusion

1. The antibody-drug conjugate comprising Trastuzumab Deruxtecan is administered in combination with TTFields to tumor cells NCI-N87 (corresponding to gastric cancer and drug target HER2). The detections of cytotoxic effect and the clonogenic effect exhibit that the treatment effect of TTFields and the antibody-drug conjugate comprising Trastuzumab Deruxtecan is more effective than that of TTFields alone or the antibody-drug conjugate comprising Trastuzumab Deruxtecan alone.
2. The antibody-drug conjugate comprising Tisotumab is administered in combination with TTFields to tumor cells CaSki (corresponding to cervical cancer and drug target TF). The detections of cytotoxic effect and the clonogenic effect exhibit that the treatment effect of TTFields and the antibody-drug conjugate comprising Tisotumab is more effective than that of TTFields alone or the antibody-drug conjugate comprising Tisotumab alone.
3. The antibody-drug conjugate comprising Sacituzumab Govitecan is administered in combination with TTFields to tumor cells SK-BR-3 (corresponding to breast cancer and drug target Trop2). The detections of cytotoxic effect and the clonogenic effect exhibit that the treatment effect of TTFields and the antibody-drug conjugate comprising Sacituzumab Govitecan is more effective than that of TTFields alone or the antibody-drug conjugate comprising Sacituzumab Govitecan alone.
4. The antibody-drug conjugate comprising Mirvetuximab Soravtansine is administered in combination with TTFields to tumor cells SK-OV-3 (corresponding to ovarian cancer and drug target FRa). The detections of cytotoxic effect and the clonogenic effect exhibit that the treatment effect of TTFields and the antibody-drug conjugate comprising Mirvetuximab Soravtansine is more effective than that of TTFields alone or the antibody-drug conjugate comprising Mirvetuximab Soravtansine alone.
5. The antibody-drug conjugate comprising Trastuzumab Deruxtecan is administered to in combination with TTFields tumor cells SK-BR-3 (corresponding to breast cancer and drug target Trop2). The detections of cytotoxic effect and the clonogenic effect exhibit that the treatment effect of TTFields and the antibody-drug conjugate comprising Trastuzumab Deruxtecan is more effective than that of TTFields alone or the antibody-drug conjugate comprising Trastuzumab Deruxtecan alone.
6. The antibody-drug conjugate comprising Trastuzumab Deruxtecan is administered in combination with TTFields to non-small cell lung cancer cells NCI-H1781 (corresponding to non-small cell lung cancer and drug target HER2). The detections of cytotoxic effect and the clonogenic effect exhibit that the treatment effect of TTFields and the antibody-drug conjugate comprising Trastuzumab Deruxtecan is more effective than that of TTFields alone or the antibody-drug conjugate comprising Trastuzumab Deruxtecan alone.

What is claimed is:

1. A method for treating a non-EGFR-expressing tumor, comprising:
(i) applying an alternating electric field to a target area, wherein the target area comprises a non-EGFR-expressing tumor or cancer cell; and
(ii) administering an effective amount of an antibody-drug conjugate.

2. A method for inhibiting the growth of a non-EGFR-expressing tumor, comprising:
(i) applying an alternating electric field to a target area, wherein the target area comprises a non-EGFR-expressing tumor or cancer cell; and
(ii) administering an effective amount of an antibody-drug conjugate.

3. A system for treating a non-EGFR-expressing tumor, comprising:
a patient information processing module and an output module;
the patient information processing module is configured to receive information on patients with the non-EGFR-expressing tumor treated with an antibody-drug conjugate, said information includes at least the medication information of the antibody-drug conjugate administered to said patients with the non-EGFR-expressing tumor; and
the output module is configured to receive information outputted by the patient information processing module and to guide the alternating electric field treatment to said patients with the non-EGFR-expressing tumor.

4. The method according to claim 1, wherein the alternating electric field has a frequency of between 100 kHz and 250 kHz.

5. The method according to claim 1, wherein the alternating electric field has a strength of between 1.6 V/cm and 1.8 V/cm.

6. The method according to claim 1, wherein the antibody-drug conjugate is selected from one or more of an antibody-drug conjugate comprising Disitamab Vedotin, an antibody-drug conjugate comprising Trastuzumab Deruxtecan, an antibody-drug conjugate comprising Trastuzumab Emtansine, DP-303c, SHR-A1811, SYD985, LCB14-0110, BDC-1001, BB-1701, TAA013, A166, an antibody-drug conjugate comprising Sacituzumab Govitecan, SKB264, an antibody-drug conjugate comprising Detopotamab Deruxtecan, BAT-8008, BL-M02D1, ESG-401, DAC-002, BAT8003, PF-06664178, an antibody-drug conjugate comprising Mirvetuximab Soravtansine, STRO-002, PRO-1184, MORAb-202, IMGN-151, BAT-8006, an antibody-drug conjugate comprising Tisotumab, an antibody-drug conjugate comprising Gemtuzumab, an antibody-drug conjugate comprising Enfortumab Vedotin-cjfv, an antibody-drug conjugate comprising Brentuximab Vedotin, an antibody-drug conjugate comprising Loncastuximab Tesirine, an antibody-drug conjugate comprising Tisotumab Vedotin, an antibody-drug conjugate Inotuzumab Ozogamicin, an antibody-drug conjugate comprising Belantamab mafodotin, and an antibody-drug conjugate comprising Cetuximab.

7. The method according to claim 1, wherein the non-EGFR-expressing tumor is selected from one or more of HER2-expressing tumors, TROP-2-expressing tumors, TF-expressing tumors, Claudin18.2-expressing tumors, TOP-1-expressing tumors, FRα-expressing tumors, CD33-expressing tumors, CD22-expressing tumors, CEACAM-5-expressing tumors, Mesothelin-expressing tumors, NECTIN4-expressing tumors, LIV1 (SLC39A6/ZIP6)-expressing tumors, ROR1-expressing tumors, GUC2C (GC-C)-expressing tumors, BCMA-expressing tumors, c-Met-expressing tumors, LRRC15-expressing tumors, LY75 (CD205)-expressing tumors, ENPP3-expressing tumors, TIM1-expressing tumors, PTK7-expressing tumors, CD142-expressing tumors, Ax1-expressing tumors, CD276-expressing tumors, CA125 (MUC16)-expressing tumors, CD70-expressing tumors, CD19-expressing tumors, CD30-expressing tumors, CD37-expressing tumors, CD46-expressing tumors, CD48-expressing tumors, CD74-expressing tumors, CD79b-expressing tumors, DLL3-expressing tumors, EpCAM-expressing tumors, ErbB3-expressing tumors, FLT3-expressing tumors, HGF/R-expressing tumors, IGF-IR-expressing tumors, CD25-expressing tumors, CD123-expressing tumors, CD56-expressing tumors, PSMA/FOLH1-expressing tumors, ROR2-expressing tumors, SLC1A5-expressing tumors, CD138-expressing tumors, TNFα-expressing tumors, TPBG-expressing tumors, TRAIL R2 (TNFRSF10B)-expressing tumors, and CD71-expressing tumors.

8. A method for treating an HER2-expressing tumor, comprising:
(i) applying an alternating electric field to a target area, wherein the target area comprises an HER2-expressing tumor or cancer cell; and
(ii) administering an effective amount of an antibody-drug conjugate, wherein the antibody-drug conjugate is selected from an antibody-drug conjugate comprising Disitamab Vedotin, an antibody-drug conjugate comprising Trastuzumab Deruxtecan, an antibody-drug conjugate comprising Trastuzumab Emtansine, DP-303c, SHR-A1811, SYD985, LCB14-0110, BDC-1001, BB-1701, TAA013, or A166.

9. The method according to claim 8, wherein the HER2-expressing tumor is breast cancer, gastric cancer, gastroesophageal junction cancer, metastatic or unresectable non-small cell lung cancer, in particular gastric cancer.

10. The method according to claim 8, wherein the antibody-drug conjugate is Enhertu®.

11. A method for treating a TROP-2-expressing tumor, comprising:
(i) applying an alternating electric field to a target area, wherein the target area comprises a TROP-2-expressing tumor or cancer cell; and
(ii) administering an effective amount of an antibody-drug conjugate, wherein the antibody-drug conjugate is selected from an antibody-drug conjugate comprising Sacituzumab Govitecan, an antibody-drug conjugate comprising Trastuzumab Deruxtecan, SKB264, an antibody-drug conjugate comprising Detopotamab Deruxtecan, BAT-8008, BL-M02D1, ESG-401, DAC-002, BAT8003, or PF-06664178.

12. The method according to claim 11, wherein the TROP-2-expressing tumor is breast cancer, in particular triple-negative breast cancer.

13. The method according to claim 11, wherein the antibody-drug conjugate is Trodelvy® or Enhertu®.

14. A method for treating an FRα-expressing tumor, comprising:
(i) applying an alternating electric field to a target area, wherein the target area comprises an FRα-expressing tumor or cancer cell; and
(ii) administering an effective amount of an antibody-drug conjugate, wherein the antibody-drug conjugate is selected from an antibody-drug conjugate comprising Mirvetuximab Soravtansine, STRO-002, PRO-1184, MORAb-202, IMGN-151, or BAT-8006.

15. The method according to claim 14, wherein the FRα-expressing tumor is ovarian epithelial cancer, fallopian tube cancer, or primary peritoneal cancer, in particular ovarian cancer.

16. The method according to claim 14, wherein the antibody-drug conjugate is Elahere®.

17. A method for treating a TF-expressing tumor, comprising:
(i) applying an alternating electric field to a target area, wherein the target area comprises a TF-expressing tumor or cancer cell; and
(ii) administering an effective amount of an antibody-drug conjugate, wherein the antibody-drug conjugate is Tivdak®.

18. The method according to claim 17, wherein the TF-expressing tumor is cervical cancer.

19. The method according to claim 8, wherein the alternating electric field has a frequency of between 100 kHz and 250 kHz.

20. The method according to claim 18, wherein the alternating electric field has a strength of between 1.6 V/cm and 1.8 V/cm.
